(19) United States
(12) Patent Application Publication
(10) Pub. No.: US 2022/0018826 A1
(43) Pub. Date: Jan. 20, 2022

(54) COLORIMETRIC PLASMONIC NANOSENSOR FOR DOSIMETRY OF THERAPEUTIC LEVELS OF IONIZING RADIATION

(71) Applicants: ARIZONA BOARD OF REGENTS ON BEHALF OF ARIZONA STATE UNIVERSITY, Scottsdale, AZ (US); BANNER HEALTH, Phoenix, AZ (US)

(72) Inventors: Kaushal Rege, Chandler, AZ (US); Karthik Subramaniam Pushpavanam, Tempe, AZ (US); Eshwaran Narayanan, Tempe, AZ (US); Stephen Sapareto, Apache Junction, AZ (US); John C. Chang, Gilbert, AZ (US)

(73) Assignees: ARIZONA BOARD OF REGENTS ON BEHALF OF ARIZONA STATE UNIVERSITY, Scottsdale, AZ (US); BANNER HEALTH, Phoenix, AZ (US)

(21) Appl. No.: 17/357,138

(22) Filed: Jun. 24, 2021

Related U.S. Application Data
(63) Continuation of application No. 15/398,590, filed on Jan. 4, 2017, now abandoned.
(60) Provisional application No. 62/275,168, filed on Jan. 5, 2016.

(51) Int. Cl. G01N 33/48; G01N 15/02

(52) U.S. Cl. CPC G01N 33/48; G01N 15/02

(57) ABSTRACT

An apparatus includes a solution including a metallic compound, a surfactant, and an acid. The solution is substantially colorless. A container holds the solution. A radiated solution is formed when the solution receives a low dose of ionizing radiation

### RELATED APPLICATIONS

This application claims priority to U.S. Provisional Application No. 62/275,168 that was filed on Jan. 5, 2016. The entire content of the applications referenced above are hereby incorporated by reference herein.

### STATEMENT REGARDING FEDERALLY SPONSORED RESEARCH

This invention was made with government support under 1403860 awarded by the National Science Foundation. The government has certain rights in the invention.

### FIELD

This disclosure relates to nanosensors for measuring therapeutic levels of ionizing radiation.

### BACKGROUND

Radiation therapy is a common primary treatment modality for multiple malignancies, including cancers of the head and neck, breast, lung, prostate, and rectum. Depending on the disease, radiation doses ranging from 20 to 70 Gy are often employed for therapeutic use. Diseased tissue and normal organ radiation sensitivities also vary. In order to maximize disease treatment relative to radiation-induced side-effects, various methods of delivery including hyperfractionation (0.5-1.8 Gy), conventional fractionation (1.8-2.2 Gy), and hypofractionation (3-10 Gy) have been explored. These delivery methods explore different regimes of radiation sensitivity in order to maximize tumor cell killing while optimizing treatment times.

Despite obvious advantages with radiotherapy, there can be significant radiation-induced toxicity in tissues. For example, radiation-induced proctitis can be a significant morbidity for patients undergoing prostate or endometrial cancer treatment. For centrally located lung cancer radiotherapy, the esophagus can be incidentally irradiated during treatments, resulting in esophagitis. In the head and neck, radiation of salivary gland or pharyngeal tumors can induce radiation-induced osteonecrosis. Another concern during radiotherapy is the motion of the patient as well as the natural peristalsis of internal organs. These issues highlight the importance of appropriately dosing the cancerous tumors while sparing the normal tissue in order to prevent significant morbidity that arises from radiation toxicity.

Despite several transformative advances since its inception in the late 19^{th} century, radiation therapy is a complex process aimed at maximizing the dose delivered to the tumor environments while sparing normal tissue of unnecessary radiation. This has led to the development of image-guided and intensity modulated radiation therapy. The process of treatment planning requires initial simulation followed by verification of dose delivery with anthropomorphic phantoms which simulate human tissue with more or less homogeneous, polymeric materials. The accuracy of the planning is measured using either anthropomorphic phantom or 3D dosimeters. During the treatment, actual dose delivery can be verified with a combination of entry, exit or luminal dose measurements. Administered in vivo doses can be measured with diodes (surface or implantable), thermoluminescent detectors (TLDs), or other scintillating detectors. However, these detectors are either invasive, difficult to handle (due to fragility or sensitivity to heat and light), require separate read-out device, or measure surface doses only. TLDs are typically laborious to operate and require repeated calibration while diodes suffer from angular, energy and dose rate dependent responses. Although MOSFETs can overcome some of these limitations, they typically require highly stable power supplies. In addition, these dosimeters require sophisticated and therefore, expensive, fabrication processes in many cases. In light of these drawbacks, there is still a need for the development of robust and simple sensors in order to assist or replace existing dosimeters that can be employed during sessions of fractionated radiotherapy.

### SUMMARY

This invention describes lipid-templated formation of colored dispersions of gold nanoparticles from colorless metal salts as a facile, visual and colorimetric indicator of therapeutic levels of ionizing radiation (X-rays), leading to applications in radiation dosimetry. The current nanosensor can detect radiation doses as low as 0.5 Gy, and exhibit a linear response for doses relevant in therapeutic administration of radiation (0.5-2 Gy). Modulating the concentration and chemistry of the templating lipid results in linear response in different dose ranges, indicating the versatility of the current plasmonic nanosensor platform.

### BRIEF DESCRIPTION OF THE DRAWINGS

FIG. 1 shows a schematic (Adapted from Pérez-Juste, J.; Liz-Marzán, L. M.; Carnie, S.; Chan, D. Y. C.; Mulvaney, P., Electric-Field-Directed Growth of Gold Nanorods in Aqueous Surfactant Solutions. Advanced Functional Materials 2004, 14 (6), 571-579.) depicting the reaction progress after addition of various components in the plasmonic nanosensor for ionizing radiation.

FIGS. 2A-2C shows a UV-Vis absorption spectra of the control (0 Gy), irradiated samples containing (FIG. 2A) C₁₆TAB, (FIG. 2B) C₁₂TAB and (FIG. 2C) C₈TAB after 7 hours.

FIGS. 3A-3E shows optical images of samples containing different C₁₆TAB and C₁₂TAB concentrations irradiated with a range of X-ray doses (Gy) (FIG. 3A) 2 mM C₁₆TAB, (FIG. 3B) 4 mM C₁₆TAB, (FIG. 3C) 10 mM C₁₆TAB, (FIG. 3D) 20 mM C₁₆TAB and (FIG. 3E) 20 mM C₁₂TAB 2 hours post irradiation.

FIG. 4. Maximum absorbance vs. radiation dose for varying concentrations of C₁₆TAB after 2 hours post irradiation. Red filled diamonds, solid line: 2 mM C₁₆TAB, Orange filled circles, dashed line: 4 mM C₁₆TAB, Green filled triangles, solid line: 10 mM C₁₆TAB, and Blue filled squares, solid line: 20 mM C₁₆TAB.

FIGS. 5A-5D shows Transmission Electron Microscopy (TEM) images of nanoparticles after exposure to ionizing (X-ray) radiation using two different lipid surfactants, 20 mM C₁₆TAB (left) and 20 mM C₁₂TAB (right). (FIG. 5A) 1 Gy, (FIG. 5B) 47 Gy, (FIG. 5C) 5 Gy and (FIG. 5D) 47 Gy.

FIGS. 6A-6B shows (FIG. 6A) An endorectal balloon with precursor solution before irradiation with X-rays and (FIG. 6B) Endorectal balloon post irradiation with 10.5 Gy X-rays.

FIGS. 7A-7B shows (FIG. 7A) Digital image showing the nanoscale precursor solution (200 μL) in microcentrifuge tubes placed along the stem outside of an endorectal balloon and (FIG. 7B) X-Ray contrast image of the phantom which shows the dose treatment plan, prostate tissue, the endorectal balloon, and the microcentrifuge tube/nanosensor location below the prostate tissue and on the endorectal balloon and (FIG. 7A)[[.]] Digital image of the plasmonic nanosensor 2 h following treatment with X-rays in the prostate phantom.

FIG. 8 shows an apparatus including a solution and a container.

FIG. 9 shows a method including mixing a metal compound with a surfactant to form a mixture and adding an acid to the mixture to form a substantially colorless solution.

FIG. 10 shows a method including mixing a fixed concentration of HAuCl₄ with a known concentration of surfactant to form a mixture and adding ascorbic acid in varying concentrations to the mixture to form a substantially colorless solution.

FIG. 11 shows a method including receiving a dose of ionizing radiation having a low ionizing dose value at a solution to form an irradiated solution including metallic nanoparticles and having an irradiated solution color and identifying the ionizing dose value by analyzing the irradiated solution color.

FIG. 12 shows a method including receiving a dose of ionizing radiation having a low ionizing dose value at a solution to form an irradiated solution including metallic nanoparticles and having an irradiated solution color and identifying the ionizing dose value by observing the irradiated solution color with a human visual system.

FIG. 13 shows a method including receiving a low dose of ionizing radiation to induce a color change in a solution including a surfactant, a metal, and an acid and observing the color change.

FIG. 14 shows a method including receiving a low ionizing radiation dose at a substantially colorless salt solution including univalent gold ions (Aul) and templating lipid micelles to form substantially maroon-colored dispersions of plasmonic gold nanoparticles.

FIG. 15 shows a method including receiving a low dose of ionizing radiation at a solution including metal salts and templating lipid micelles to form colored dispersions from nanoparticle formations in the solution.

FIG. 16 shows a method including receiving a low dose of ionizing radiation at a solution including metal salts and templating lipid micelles to form metal nanoparticles from the metal salts.

FIG. 17 shows a method that includes delivering a therapeutic dose of radiation to an animal and a dosimeter and measuring the therapeutic dose of radiation at the dosimeter, the dosimeter including a solution having metallic nanoparticles after receiving the therapeutic dose of radiation.

FIG. 18 shows a method that includes delivering a therapeutic radiation dose having a radiation value to a human and a solution including a surfactant, a metal, and an acid to form a radiated solution having a color and determining the radiation value by analyzing the color.

FIG. 19 shows UV-Visible spectral profiles of (A) HAuCl₄, (B) HAuCl₄ (0.196 mM)+C₁₆TAB (20mM), (C) HAuCl₄ (0.196 mM)+C₁₆TAB (20 mM)+Ascorbic Acid (5.88 mM) and (D) HAuCl₄ (0.196 mM)+Ascorbic Acid (5.[[88 mM)AA).

FIGS. 20A-20B shows (FIG. 20A) UV-Vis spectra of varying ascorbic acid volumes along with gold and C₁₆TAB irradiated at 47 Gy and (FIG. 20B) maximum absorbance values of samples containing varying concentrations of ascorbic acid denoted as [AA].

FIGS. 21A-21C shows absorbance spectra of (FIG. 21A) gold salt (0.196 mM) (FIG. 21B) gold salt (0.196 mM)+C₁₆TAB (20 mM) (FIG. 21C) gold salt (0.196 mM)+C₁₂TAB (20 mM).

FIGS. 22A-22C shows kinetics of gold nanoparticle formation following exposure to different doses of ionizing radiation (0-47 Gy) for (FIG. 22A) C₁₆TAB, (FIG. 22B) C₁₂TAB and (FIG. 22C) C₈TAB.

FIG. 23 shows maximum absorbance vs. radiation dose (Gy) after 2 hours of X-ray irradiation. C₁₆TAB (red filled squares, solid line) and C₁₂TAB (orange open circles, dotted line) surfactants.

FIG. 24 shows intensity ratio of 1337/1334 as a function of surfactant concentration is used to determine the critical micellar concentration.

FIGS. 25A-25C shows absorbance spectra of precursor monovalent gold salt solutions under conditions of no radiation (i.e. 0 Gy) in presence of different concentrations of (FIG. 25A) C₁₆TAB and (FIG. 25B) C₁₂TAB (FIG. 25C) C₈TAB recorded after 10 minutes of incubation.

FIGS. 26A-26D shows Maximum Absorbance vs. Wavelength for different concentrations of C₁₆TAB after a duration of 2 hours post irradiation (FIG. 26A) 2 mM (FIG. 26B) 4 mM (FIG. 26C) 10 mM (FIG. 26D) 20 mM.

FIGS. 27A-27B shows (FIG. 27A) Hydrodynamic diameter vs. radiation dose and (FIG. 27B) Hydrodynamic diameter vs. radiation dose on a log₁₀ scale.

FIGS. 28A-28D shows transmission electron microscopy (TEM) images of anisotropic nanostructures (FIG. 28A) dendritic and (FIG. 28C) nanowire-like structures formed in case of C₁₂TAB at 5 Gy X-ray radiation dose and images (FIG. 28B) and (FIG. 28D) show magnified images of the highlighted regions inside red box from Figures (FIG. 28A) and (FIG. 28C).

FIGS. 29A-29G shows Transmission Electron Microscopy (TEM) images of nanoparticles formed after exposure to ionizing (X-ray) radiation using the following conditions of C₁₆TAB: (FIG. 29A) 10 mM and 5 Gy, (FIG. 29B) 10 mM and 47 Gy, (FIG. 29C) 4mM and 5 Gy, (FIG. 29D) 4 mM and 15 Gy, (FIG. 29E) 2 mM and 0.5 Gy, (FIG. 29F) Magnified image of highlighted area of E, and (FIG. 29G) 2 mM and 2.5 Gy.

FIG. 30 shows a digital image showing the phantom irradiation set up on the linear accelerator at Banner MD Anderson.

### DESCRIPTION

Facile radiation sensors have the potential to transform methods and planning in clinical radiotherapy. Below are described results of studies on a colorimetric, liquid-phase nanosensor that can detect therapeutic levels of ionizing radiation. X-rays, in concert with templating lipid micelles, were employed to induce the formation of colored dispersions of gold nanoparticles from corresponding metal salts, resulting in a easy to use visible indicator of ionizing radiation.

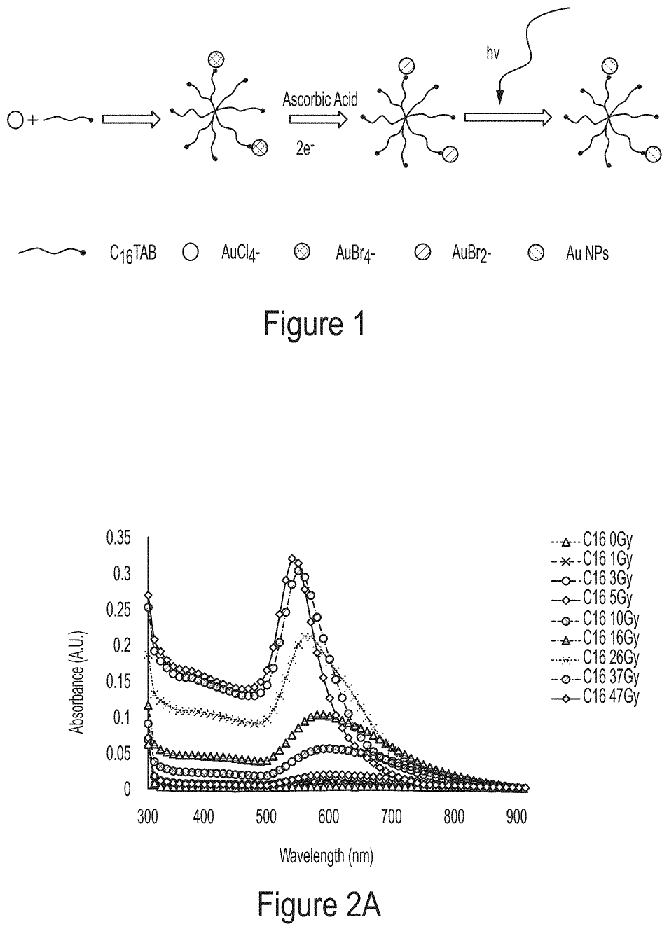

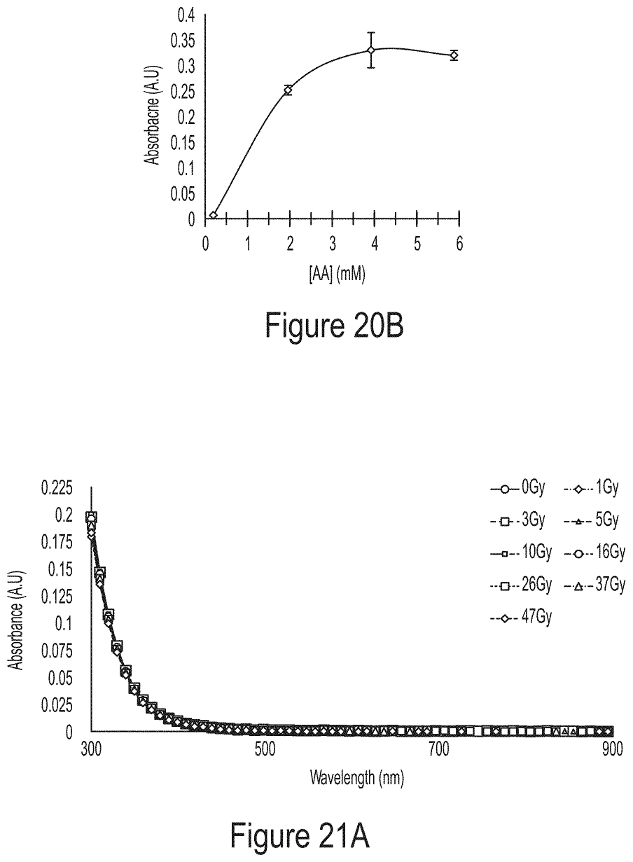

The novel plasmonic nanosensor employs a colorless metal salt solution comprising a mixture of auric chloride (HAuCl₄), L-Ascorbic acid (AA) and cetyl (C₁₆), dodecyl (C₁₂), or octyl (C₈) trimethylammonium bromide (C_{x; x=16/12/8}TAB) surfactant molecules (FIG. 1; please see the Experimental Section for more details). In brief, CₓTAB and HAuCl₄ were first mixed leading to the formation of Au^{III}Br₄⁻. HAuCl₄ shows a prominent peak at 340 nm which shifts to 400 nm after addition of C₁₆TAB, likely due to the exchange of a weaker chloride ion by a stronger bromide ion (FIGS. 19A and 19B, Supporting Information section). The shift in absorption peak can also be seen visually as a color change from yellow to orange. Subsequent addition of ascorbic acid turns the solution colorless with no observable peaks between 300 and 999 nm (FIG. 19C, Supporting Information section). Ascorbic acid reduces Au(III) to Au(I) in a two-electron, step-reduction reaction. It has been shown that addition of up to 5 molar equivalent excess ascorbic acid does not result in the formation of zerovalent gold or Au(0) species, which can be partly attributed to the lower oxidation potential of the acid in presence of C₁₆TAB. This mixture of CₓTAB, ascorbic acid, and HAuCl₄ is employed as the precursor solution for radiation sensing. However, a characteristic peak in the range of 500-600 nm corresponding to gold nanoparticles is observed if ascorbic acid directly reacts with the gold salt in the absence of C₁₆TAB (FIG. 19D, Supporting Information section), indicating spontaneous formation of nanoparticles in absence of the surfactant under the conditions employed.

First, attempts were made to convert trivalent gold to its univalent state, since the reduction of Au(I) to Au(0) is thermodynamically favored over the reduction of Au(III) to Au(0), due to a higher standard reduction potential of the former. Au(I) has an electronic configuration of 4f¹⁴5d¹⁰, and requires a single electron for conversion (reduction) to Au(0). This formation of zerovalent gold or Au(0) is a prerequisite step for nanoparticle formation. In the current plasmonic nanosensor, the electron transfer required for converting Au(I) to Au(0) is facilitated by splitting water into free radicals following exposure to ionizing radiation (X-rays).

Water splitting by ionizing radiation generates three key free radicals, two of which, e⁻ and H., are reducing, and the other (.OH.) oxidizing in nature. Excess ascorbic acid is an antioxidant capable of removing the detrimental (oxidizing) OH. radicals generated in the system. CₓTAB surfactants were employed due for their ability to template gold nanoparticles. These three species, namely ascorbic acid, CₓTAB, and gold salt, form the key constituents of the current plasmonic nanosensor for ionizing radiation.

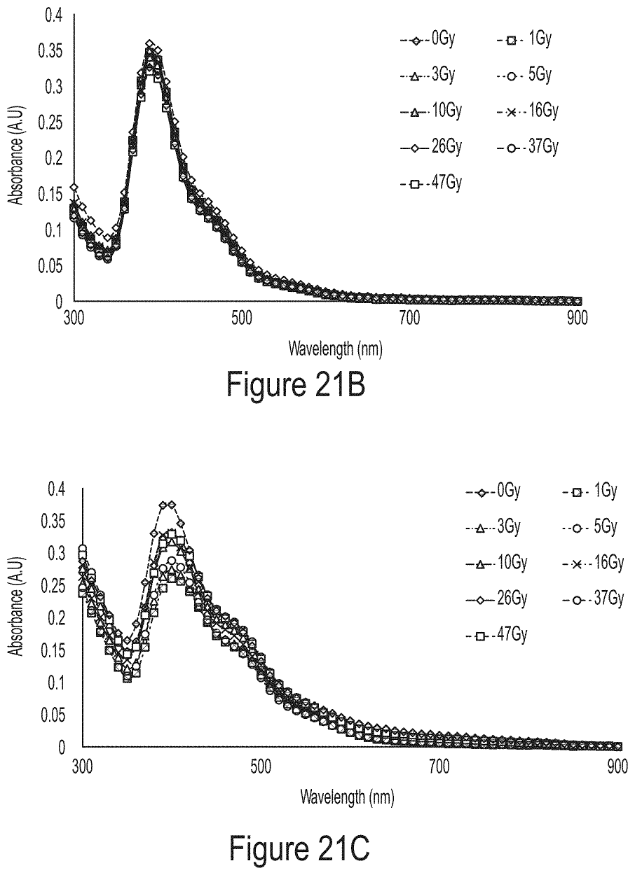

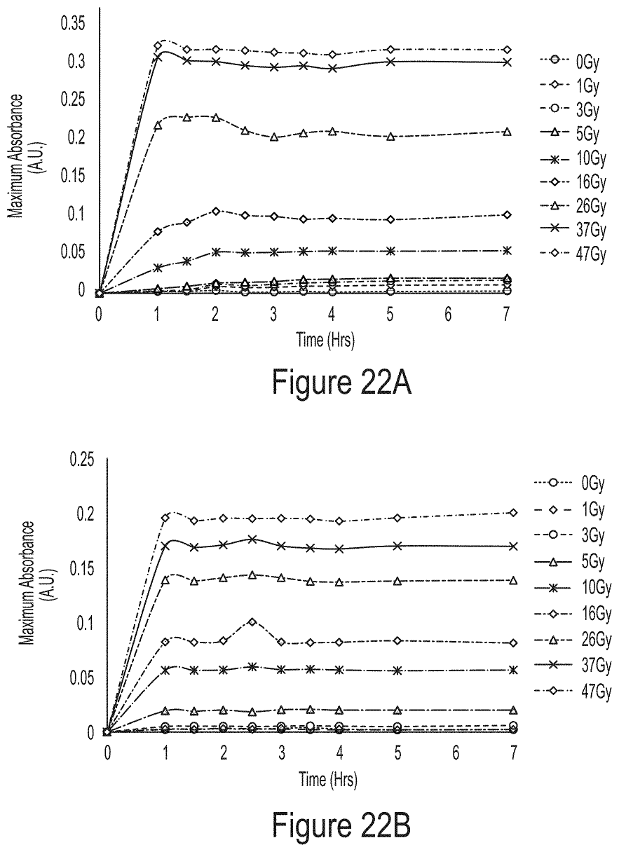

First, the concentration of ascorbic acid (AA) was optimized in the presence of the surfactant (C₁₆TAB) and gold salt employed in the plasmonic nanosensor; the maximal dose of 47 Gy was delivered in order to study the effect of ascorbic acid on nanoparticle formation (FIGS. 20A-20B, Supporting Information section). A marked increase in nanoparticle formation is observed when excess AA is used and it reaches saturation when 600 μL of 0.01 M (4 mM AA) is employed; similar levels of nanoparticle formation are seen when 900 μL of 0.01 M (5.88 mM AA) are employed. Although saturation was observed when 600 μL of AA were used, 5.88 mM AA was used for all subsequent experiments in order to ensure adequate quenching of the detrimental OH. radicals which otherwise adversely affects the yield of nanoparticles generated. Control experiments with (1) gold salt (HAuCl₄) alone, (2) gold salt+C₁₆TAB and (3) gold salt+C₁₂TAB were also carried out in presence of different X-ray doses, but in absence of ascorbic acid. Absorbance profiles of the samples were measured after 7 hours and the absence of peaks from 500-900 nm indicated the absence of plasmonic (gold) nanoparticles (FIGS. 21A-21C, Supporting Information section).

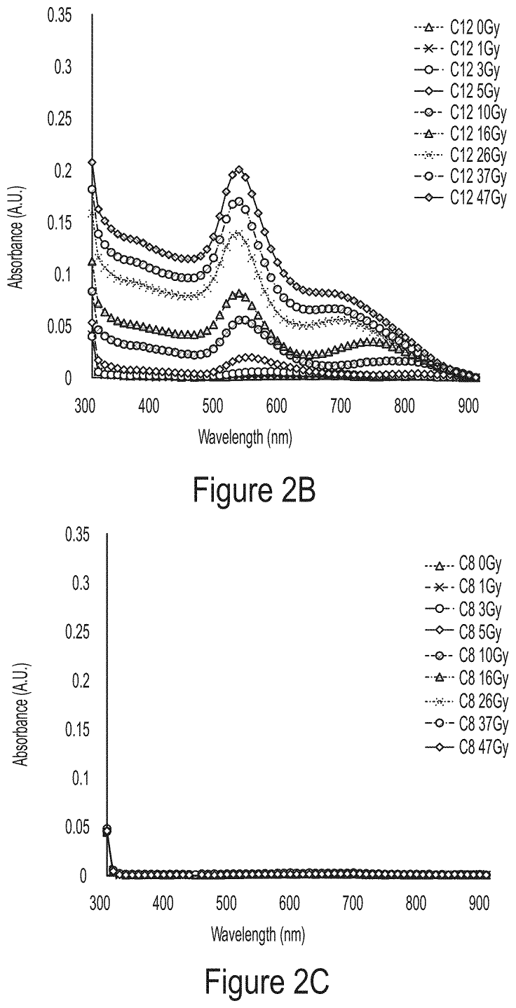

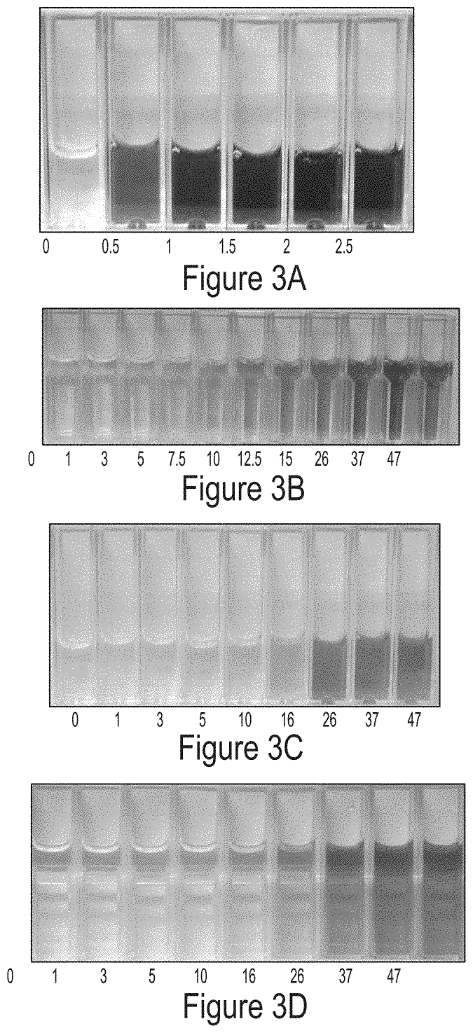

Next, the efficacy of three cationic surfactants, C₈TAB C₁₂TAB, and C₁₆TAB was investigated, for inducing nanoparticle formation in presence of different doses of ionizing radiation (FIGS. 2A-2C). All three surfactants have trimethyl ammonium moieties as the head group and bromide as the counter ions; only the lipid chain length was varied as C₈, C₁₂, and C₁₆ in these molecules. As stated previously, a large number of e⁻_{aq} and H. radicals are generated following exposure of the solution to X-rays which facilitate the conversion of Au⁺ ions to their zerovalent Au⁰ state. The Au⁰ species act as seeds upon which further nucleation and coalescence occurs. This, in turn, leads to an increase in size and eventual formation of nanoparticles, which are stabilized by surfactant molecules. Formation of these plasmonic nanoparticles imparts a burgundy/maroon color to the dispersion; the intensity of the color increases with an increase in radiation dose applied (FIGS. 3A-3E).

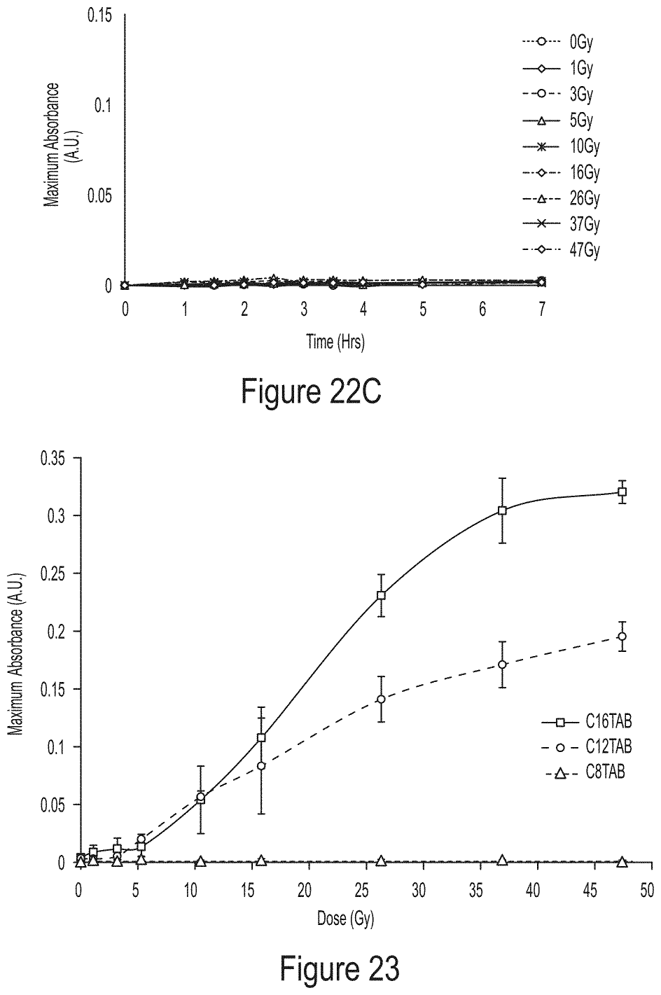

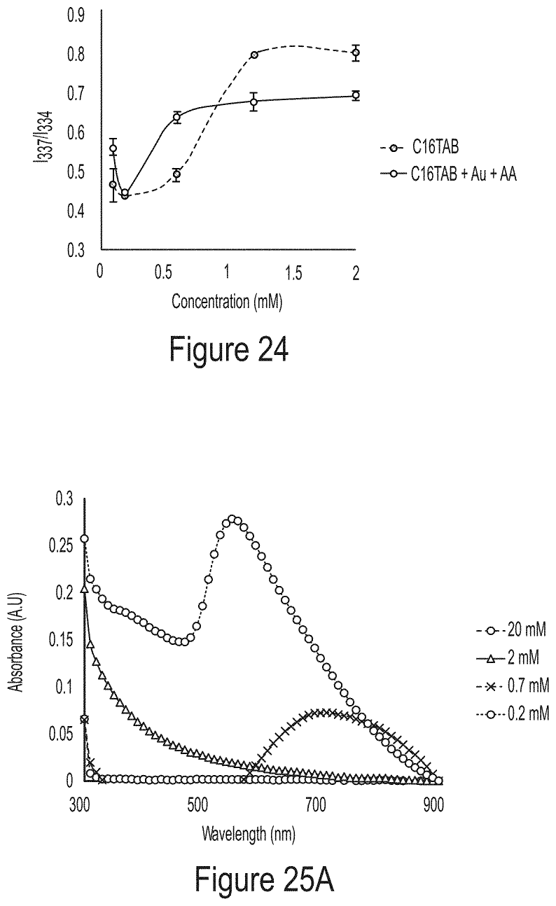

Nanoparticle formation was seen as early as 1 h following irradiation in many cases, although 2 h were required for samples irradiated with lower doses (1, 3 and 5 Gy) (FIGS. 22A-22C, Supporting Information section). No significant differences in absorbance intensity were observed thereafter until a period of 7 hours, which was the maximum duration investigated in these cases. Nanoparticle formation was observed at radiation doses as low as 1 Gy, which is well within the range of doses employed for radiotherapy. While C₁₆TAB or C₁₂TAB were effective at templating nanoparticle formation even at low doses (1-5 Gy), C₈TAB did not show any propensity for templating nanoparticle formation even at the highest radiation dose (47 Gy) employed. C₁₂TAB-templated gold nanoparticles exhibited unique spectral profiles under ionizing radiation; two spectral peaks—one between 500 and 550 nm and another between 650 and 800 nm—were seen (FIG. 2B). This is in contrast to C₁₆TAB which exhibited only a single peak between 500 and 600 nm (FIG. 2C). Finally, the linear response for C₁₆TAB was significantly more pronounced than that for C₁₂TAB (FIG. 23).

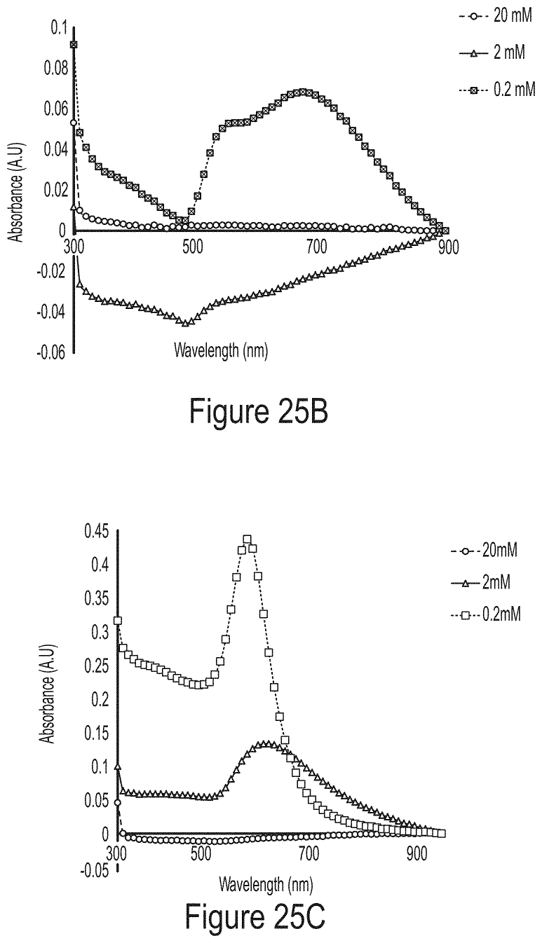

The critical micelle concentration (CMC) of C₁₆TAB is reported to be approximately 1 mM. Using the pyrene fluorescence assay, we determined the CMC of C₁₆TAB in the nanosensor precursor solution (i.e. gold salt and ascorbic acid in water) to be ˜0.7±0.1 mM, which is slightly lower than ˜1.2±0.02 mM in THIS solvent (FIG. 24, Supporting Information section). Pre-micellar aggregates are thought to exist when C₁₆TAB concentration is lower than 7.4 mM, while stable micelles are observed at higher concentrations of the lipid surfactant. One hypothesis is that increasing the ratio of the metallic species (Au⁺) to the aggregate (pre-micellar/micellar) C₁₆TAB species would lead to greater propensity for nanoparticle formation upon exposure to ionizing radiation and therefore increased sensitivity of the resulting nanosensor at lower radiation doses. Based on the hypothesis that the number of aggregate species increases with lipid concentration, lower concentrations of C₁₆TAB (2 mM, 4 mM and 10 mM) was investigated, while keeping the gold and ascorbic acid concentration constant.

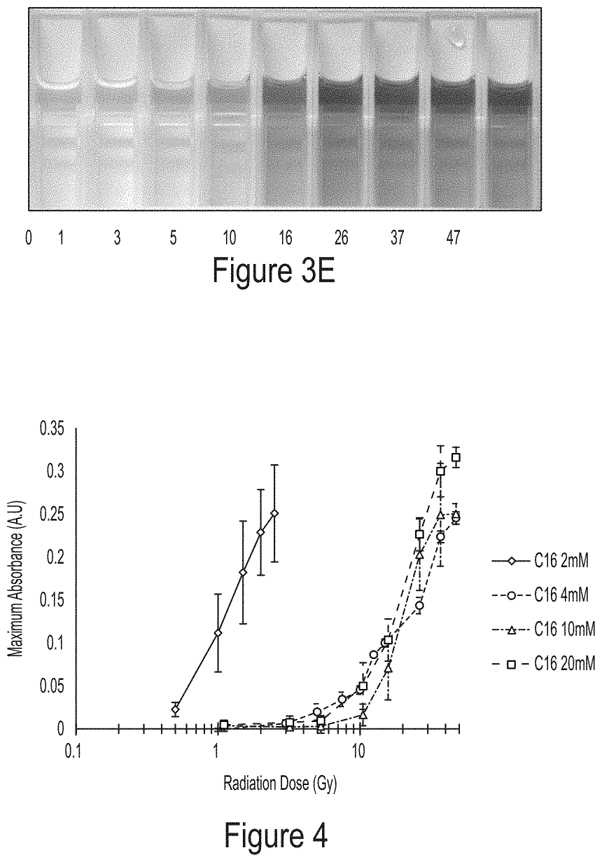

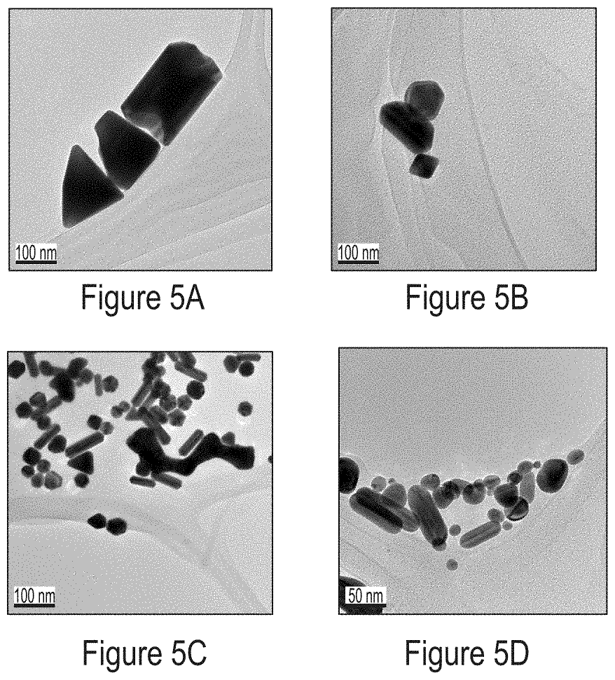

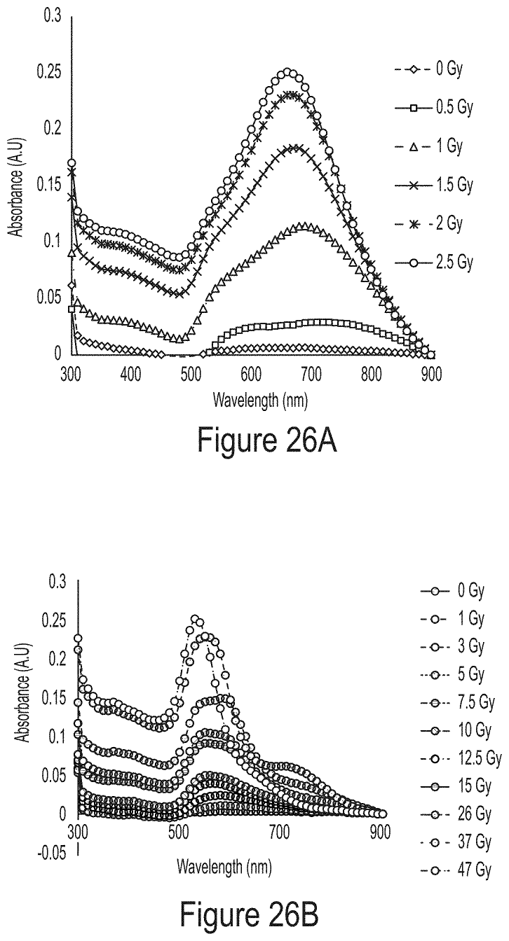

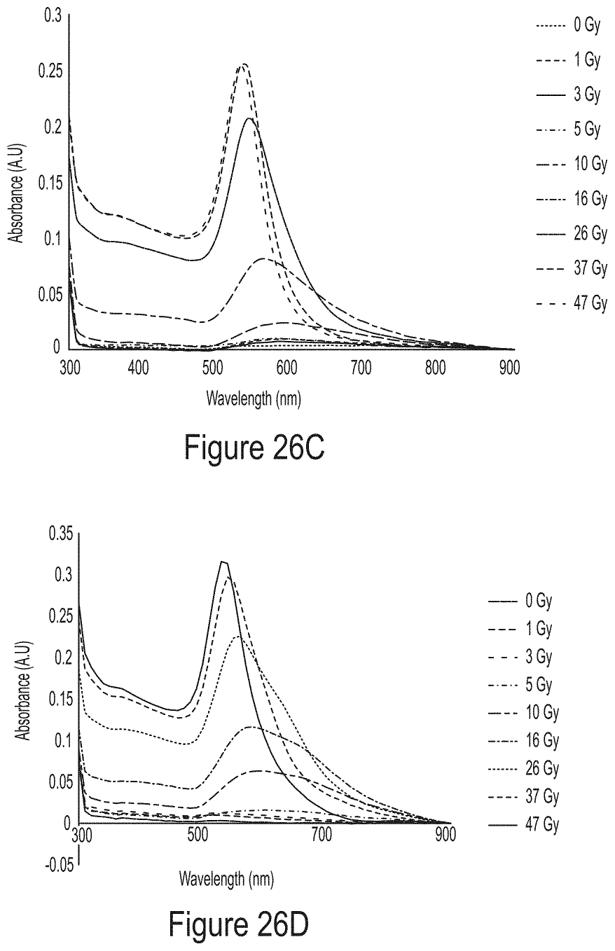

Use of C₁₆TAB concentrations at and below the CMC (i.e. 0.7 and 0.2 mM) resulted in spontaneous formation of gold nanoparticles in absence of ionizing radiation; gold nanoparticle formation can be seen by the characteristic absorbance peak of the dispersion in FIGS. 25A-25C, Supporting Information Section. However, the propensity for spontaneous nanoparticle is significantly reduced or lost at concentrations above the CMC. A distinct color change can be observed for radiation doses as low as 0.5 Gy for the lowest concentration of C₁₆TAB above the CMC investigated (FIGS. 3A and 26A-26D, Supporting Information section). A linear response was observed for radiation doses ranging from 0.5 to 2 Gy under these conditions (FIGS. 5A-5D). As the concentration of C₁₆TAB increases, the radiation dose required to template nanoparticle formation also increases (FIGS. 4 and 26A-26D, Supporting Information section). Furthermore, the color of the nanoparticle dispersion formed is significantly different in cases of 2 mM (blue-violet) C₁₆TAB compared to that observed in cases of 4 mM (bluish-red), 10 mM (red/pink) and 20 mM (burgundy/maroon) C₁₆TAB, indicating different sizes of nanoparticles under these conditions. While it is most desired that the nanosensor is sensitive to therapeutic doses used in conventional and hyperfractionated radiotherapy (˜0.5-2.2 Gy), these results indicate that the response of the plasmonic nanosensor can be tuned by simply modifying the concentration of the lipid surfactant.

Visual colorimetric sensors possess advantages of convenience and likely, cost, over those that employ fluorescence changes or electron spin resonance measurements for detecting ionizing radiation. The current plasmonic nanosensor shows increasing color intensity with increasing radiation dose (FIGS. 2A-2C and 3A-3E). The increase in color intensity with radiation dose is reflected in an increase in maximal (peak) absorbance values, which in turn, are surrogates for the concentrations of nanoparticles formed in the dispersion. Key features of gold nanoparticle absorbance spectra include the shape of the surface plasmon resonance band and the position of the maximal (peak) absorption wavelength. The width of the spectral profiles at lower doses signifies a somewhat polydisperse population of the nanoparticles (FIGS. 2A-3C and FIGS. 26A-26D Supporting Information section). The absorbance peaks are red-shifted with decreasing radiation doses, suggesting an increase in particle size under these conditions compared to those obtained at higher doses.

Free radicals generated upon radiolysis are thought to be localized in finite volumes called spurs. These spurs can expand, diffuse, and simultaneously, react, leading to the formation of molecular products. These highly reactive free radicals have very short lifetimes of ˜10⁻⁷-10⁻⁶ s at 25° C. Reaction volumes consisting of nanoscale features can facilitate enhanced reaction kinetics and ensure efficient utilization of these free radicals for the formation of nanoparticles. In case of the current plasmonic nanosensor, this was achieved by the use of amphiphilic molecules that self-assemble into micelles above their respective critical micellar concentrations (CMCs). A strong interaction is possible between the positively charged head group of the lipid surfactant micelles and the negatively charged AuCl₄⁻ ions (FIG. 1). This interaction can lead to incorporation of AuCl₄⁻ ions in the water-rich Stern layer leading to the formation of a ‘nanoreactor’. However, spontaneous formation of nanoparticles (i.e. in absence of ionizing radiation) was seen when concentrations of C₁₆TAB were lower than the CMC (FIGS. 25A-25C Supporting Information section). One hypothesis is that spontaneous nanoparticle formation observed at lower concentrations of the surfactant is likely due to negligible steric hindrance between the surfactant and ascorbic acid; absence of these barriers results in nanoparticle growth which can be spectroscopically observed. It is only when the concentrations of C₁₂TAB and C₁₆TAB are higher than the CMC, that no spontaneous formation of gold nanoparticles is seen, and ionizing radiation is required to induce nanoparticle formation. This, therefore, acts as the functional principle behind the current plasmonic nanosensor. Of the three lipid surfactants, only the concentration of C₈TAB was significantly below its CMC value (130 mM), while the concentrations employed were significantly higher than the CMCs of C₁₂TAB (CMC=15 mM) and C₁₆TAB (CMC=1 mM). In the case of C₈TAB, there is an absence of these “nanoreactors”, which may explain lack of nanoparticle formation under these conditions. These observations suggest that interplay between surfactant chemistry and aggregation state determine nanoparticle formation by lipid-based surfactant molecules.

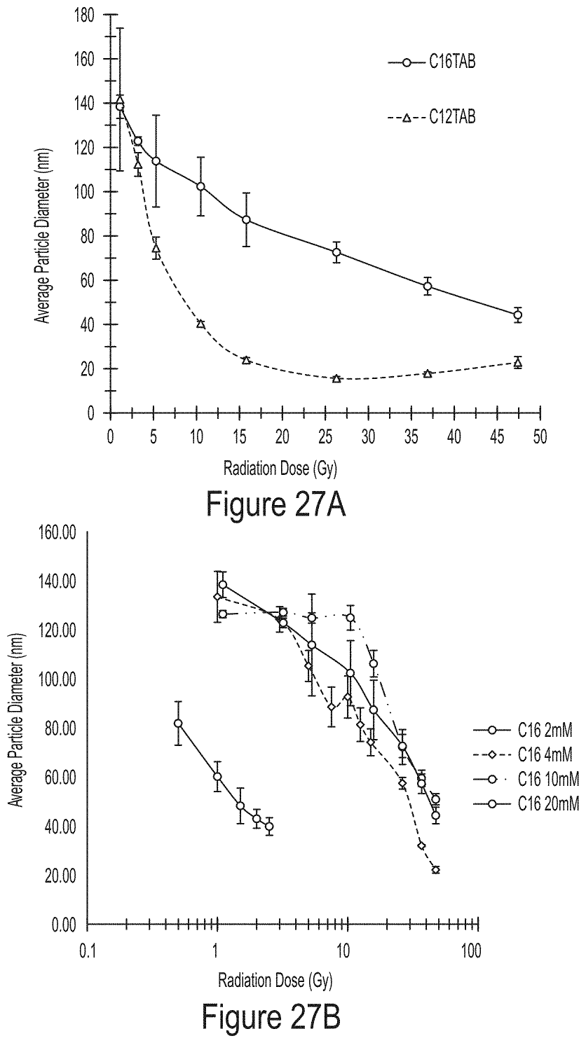

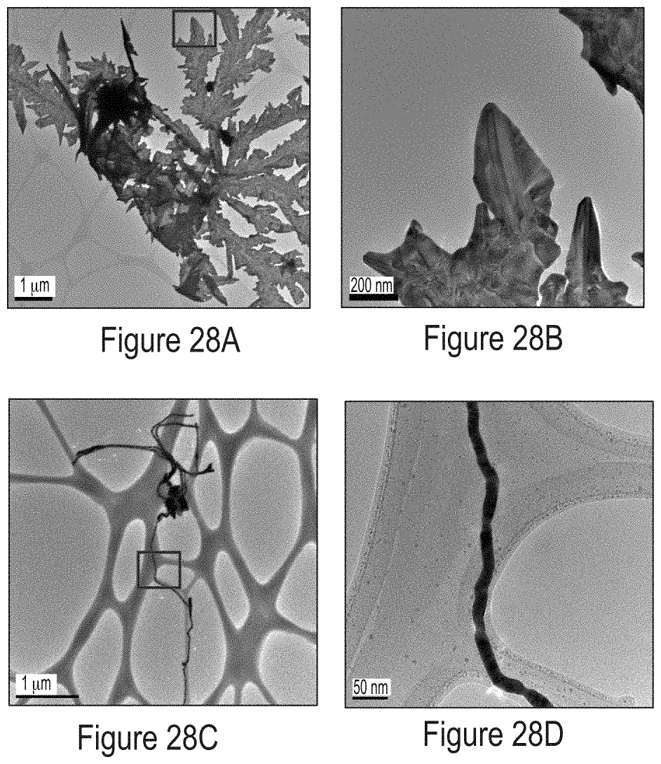

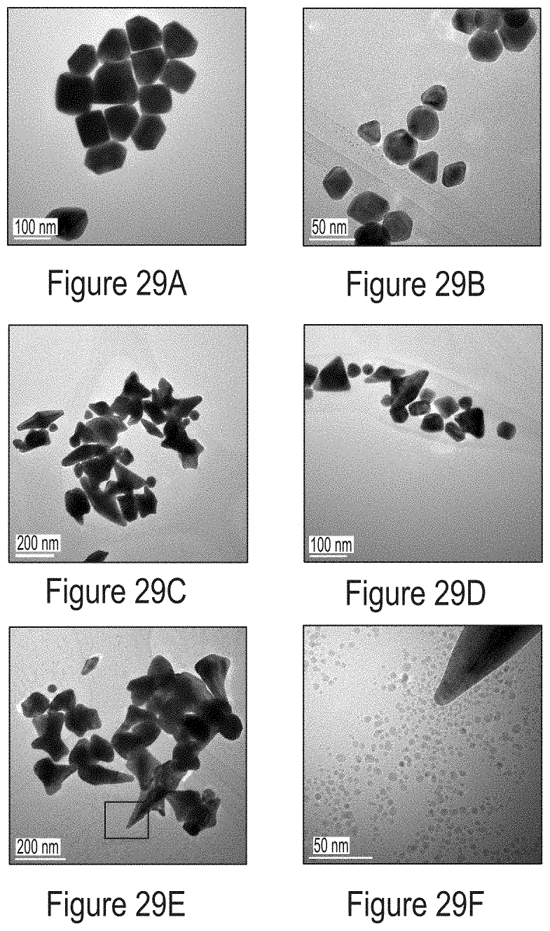

Nanoparticles formed in presence and absence of ionizing radiation were characterized for their morphology and hydrodynamic diameter using transmission electron microscopy (TEM; FIGS. 5A-5D, and FIGS. 28A-28D and 29A-29G, Supporting Information section) and dynamic light scattering (FIGS. 27A-27B, Supporting Information section), respectively. While C₁₆TAB-templated nanoparticles showed a single maximal absorption peak (at ca. 520 nm), C₁₂TAB-templated nanoparticles showed two peaks: one at ca. 520 nm (visual region) and another at ca. 700 nm (near infrared or NIR region; FIG. 2B), particularly at higher doses of ionizing radiation. TEM images indicated that a mixture of spherical and rod-shaped nanoparticles was observed at the higher radiation doses (47 Gy) in case of C₁₂TAB as the templating surfactant (FIG. 5D). This explains the absorption spectral profile with peaks in both, the visual and near infrared range of the spectrum in case of nanoparticles templated using C₁₂TAB (FIG. 2B). A significant decrease in the near infrared absorption peak is observed at lower X-ray doses. Although the spectral profile indicates formation of gold nanospheres, we observed an ensemble of unique anisotropic (dendritic and nanowire) structures (FIGS. 28A-28D, Supporting Information section). Such structures were not observed at similar X-ray doses in case of C₁₆TAB as the templating surfactant.

The growth of gold nuclei from zerovalent gold species proceeds through continuous diffusion of unreacted metal ions and smaller seeds onto the growing nanocrystal surface. This, in turn, is governed by electrostatic interactions between the cationic micelle loaded with gold seeds and unreacted metal ions. In this case, it is likely that the gold nanoparticles aggregate more rapidly in situ due to the strong hydrophobic nature of the long of C₁₆TAB chains, leading to the formation of quasi-spherical nanoparticles and not anisotropic nanostructures.

TEM images indicated a reduction in the size of the metal nanoparticles with increasing radiation dose. Dynamic light scattering (DLS) studies on irradiated samples (FIGS. 27A-27B, Supporting Information section and Table 3, Supporting Information section) indicated a linear decrease in nanoparticle hydrodynamic diameters with increases in X-ray dose, which is in good agreement with information from TEM images. High radiation doses generate a larger number of free radicals in comparison to lower radiation doses, which can lead to the reaction with and therefore, consumption of a higher number of metal ions. This leads to the formation of a higher concentration of zerovalent gold species in comparison to samples irradiated at lower doses. These unstable Au(0) seeds grow and are eventually capped by the cationic surfactant resulting in smaller sized nanoparticles. In contrast, at lower doses of ionizing radiation, the ratio of concentration of Au(0) to Au(I) is likely smaller. It is possible that unreacted metal ions coalesce with the smaller population of gold seeds and in turn lead to the formation of nanoparticles with larger diameters.

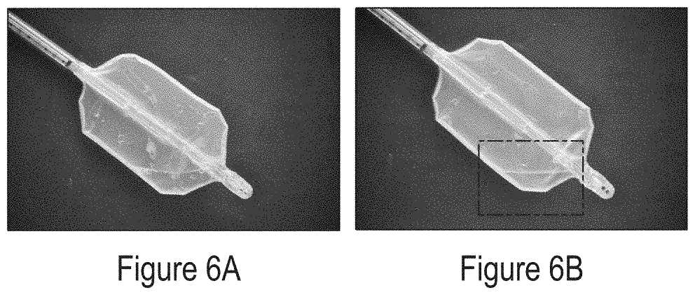

The translational potential of a plasmonic nanosensor for detecting X-ray radiation was investigated under conditions that simulate those employed in human prostate radiotherapy. Endorectal balloons are typically used for holding the prostate in place and for protecting the rectal wall during radiotherapy treatments in humans. The efficacy of the plasmonic nanosensor was evaluated in these balloons ex vivo; no studies on human patients were carried out. 1.5 ml of the precursor solution (C₁₆TAB (20mM)+AA+HAuCl₄) was incorporated into endorectal balloons as shown in FIG. 6A. The nanosensor precursor solution was subjected to two clinically relevant doses of 7.9 and 10.5 Gy (n=3). The absorbance of the plasmonic nanosensor, which changes color in the balloon itself (e.g. light pink color seen in FIG. 6B for a balloon subjected to a radiation dose of 10.5 Gy) was employed to determine the radiation dose delivered to the balloon. A calibration curve between 5 and 37 Gy from the plot between maximum absorbance and radiation dose after 7 hours was employed to determine the radiation dose delivered. Doses of 8.51±1.73 Gy and 7.85±2.05 Gy were calculated from the calibration curve for 10.5 Gy and 7.9 Gy respectively. Due to the nonlinearity of the curve below 5.3 Gy, the control (0 Gy) showed a value 4.38±0.41 Gy (n=3) when the calibration equation was employed, indicating that the operating region of the plasmonic nanosensor, with a CTAB concentration of 20 mM, is between 5 and 37 Gy and is not reliable for lower doses of radiation for CTAB concentrations of 20 mM (Table 1).

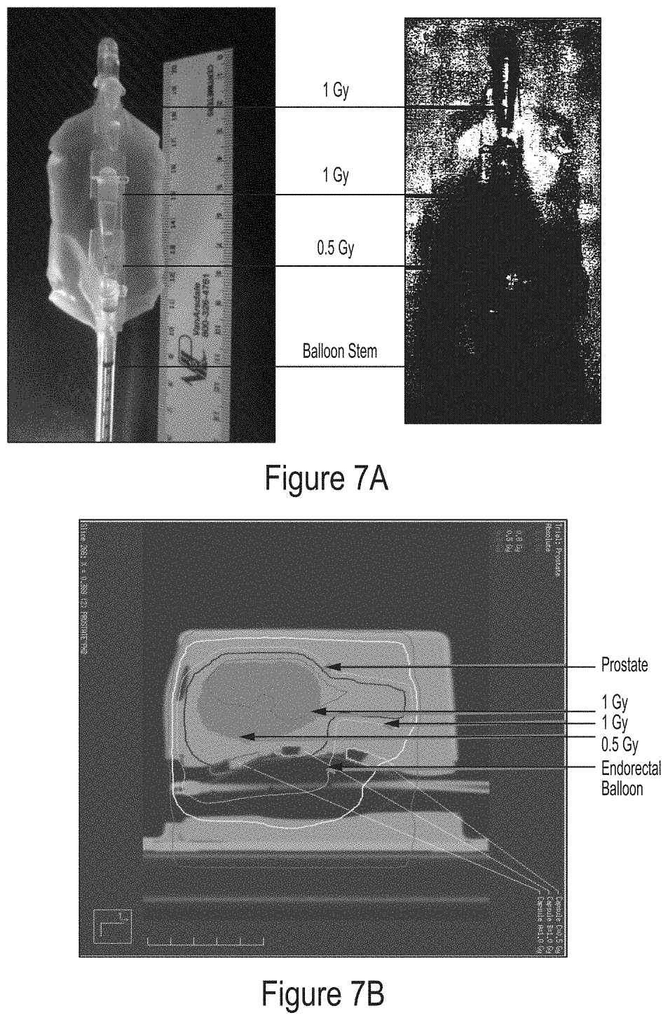

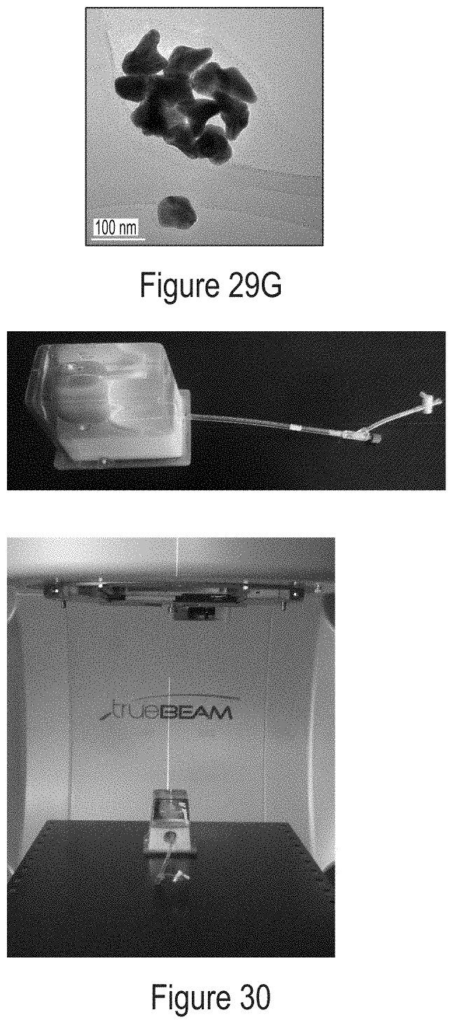

Based on the above findings in the endorectal balloon, the detection efficacy of the plasmonic nanosensor in a phantom that is employed to simulate prostate radiotherapy treatments was investigated. In these studies, 200 μL of the precursor solution (C₁₆TAB (2 mM)+AA+HAuCl₄) was filled in microcentrifuge tubes, which were then taped to the outside surface of an endorectal balloon such that they were aligned along the stem (FIG. 7A). The lower concentration of C₁₆TAB was used, since this concentration results in detection between 0.5-2 Gy (FIGS. 3A-3E top panel). The prostate phantom, with the endorectal balloon placed under the simulated prostate tissue, was irradiated based on a treatment plan described in the Experimental section and shown in FIGS. 30 and 7B. The prostate itself was irradiated with 1 Gy, while the dose fall off at the end was 0.5 Gy (n=3; FIG. 7B). Thus, two microcentrifuge tubes (capsules 1 and 2) along the stem of the balloon just below the prostate were subjected to 1 Gy, while the third one (capsule 3) outside the balloon was subjected to 0.5 Gy. This set up was employed in order to obtain spatial information on the delivered dose along the rectal wall in the tissue phantom.

Optical images (FIG. 7A) clearly indicate the formation of violet colored dispersions for capsules 1 and 2, while a dispersion of lighter intensity can be seen in capsule 3. The absorbance of the dispersions were measured 2 h following exposure to radiation, and a calibration curve was employed to estimate the radiation dose as indicated by the radiation sensor. Table 2 shows a comparison of the actual dose delivered and the dose estimated from the calibration of the plasmonic nanosensor. The plasmonic nanosensor indicates that capsules 1 and 2 received doses of 1.20±0.11 Gy and 1.17±0.16 Gy, respectively, while capsule 3 received a dose of 0.49±0.04 Gy (Table 2). These are highly reasonable estimates of the actual doses received by the capsules in the tissue phantom, and can be employed to obtain spatial information on the radiation dose delivered. Taken together, the results indicate the utility of the plasmonic nanosensor in as a simple detection system in simulated clinical settings.

The application discloses an easy to use, versatile and powerful nanoscale platform for dosimetry of therapeutically relevant doses of radiation. This method involves readily available chemicals, is easy to visualize due to the colorimetric nature of detection, and does not need expensive equipment for detection. While a ‘yes/no’ determination may be made by the naked eye, only an absorbance spectrophotometer is required for quantifying the radiation dose. A visible color change also ensures the ease of detecting the radiation dose with the naked eye. It was found that both, C₁₂TAB and C₁₆TAB were able to function as templating molecules in the plasmonic nanosensor at concentrations above their critical micelle concentration (CMC). The sensitivity of the sensor to lower radiation doses is enhanced by modifying the concentration of C₁₆TAB, thus making this a highly versatile platform for a variety of applications. Apart from the surfactants used a list of other potential surfactants which could be employed are listed in the Table 4. The chemicals included in the list along with their derivatives are potential chemicals which could be used along with our sensor in its current form or in any other formulation. The metal ions used is not limited to gold. Any species either metallic or non-metallic can be used along with the sensor in its current form or in any other formulation. To name a few, ions of cobalt, iron, silver could be potential replacement for the proof of concept gold employed .The utility of the plasmonic nanosensor was demonstrated in translational applications; the plasmonic nanosensor was able to detect the delivered radiation dose with satisfactory accuracy when placed in an endorectal balloon ex vivo. In addition, the nanosenor was able to detect doses as low as 0.5 Gy and was able to report on the spatial distribution of radiation dose delivered when investigated using an endorectal balloon placed in a prostate tissue phantom. The translational application of such a dosimeter can help therapists with treatment planning and potentially enhance selectivity and efficacy of treatment. Apart from the medical field, this sensor could be employed where there is a need to detect ionizing radiation directly or indirectly.

### Apparatus

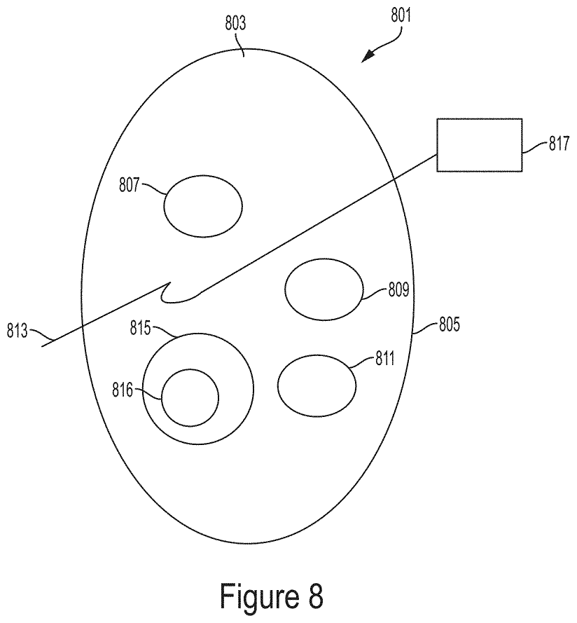

FIG. 8 shows an apparatus 801 including a solution 803 and a container 805. A solution is a substantially homogeneous mixture of two or more substances, which may be solids, liquids, gases, or a combination of solids, liquids or gases. The solution 803 includes a metallic compound 807, a surfactant 809, and an acid 811. A metallic compound is compound that contains one or more metal elements. An exemplary metallic compound suitable for use in connection with apparatus 801 includes auric chloride (HAuCl₄). A surfactant is a compound that lowers the surface tension (or interfacial tension) between two liquids. Exemplary surfactants suitable for use in connection with the apparatus 801 include cetyl trimethylammonium bromide (C₁₆TAB) and dodecyl trimethylammonium bromide (C₁₂TAB). In some embodiments, the apparatus 801 includes a surfactant 809 that has a critical micelle concentration of about 0.7+0.1 nm. The critical micelle concentration (CMC) is defined as the concentration of surfactants above which micelles form and all additional surfactants added to the system go to micelles. An acid is a chemical substance whose aqueous solutions are characterized by an ability to react with bases and certain metals to form salts. An exemplary acid 811 suitable for use in connection with the apparatus 801 includes L-ascorbic acid.

The container 805 holds the solution 803. Containers 805 suitable for use in connection with the apparatus 801 are not limited to particular types of containers. In some embodiments, the container 805 includes an endorectal balloon.

In operation, the solution 803 of the apparatus 801 receives a low dose of ionizing radiation 813 to form a radiated solution 815. In some embodiments, the irradiated solution 815 includes a plasmonic nanoparticle 816. A plasmonic nanoparticle is a particle whose electron density can couple with electromagnetic radiation having wavelengths that are larger than the particle due to the nature of the dielectric-metal interface between the medium and the particles.

In some embodiments, the low dose of ionizing radiation 813 is not limited to a particular radiation value. In some embodiments, the low dose of ionizing radiation 813 includes a therapeutic range of values such as between about 0.5 Gy and about 2.0 Gy. In some embodiments, the low dose of ionizing radiation 813 includes a range of values of between about 1.7 Gy and about 2.2 Gy. In some embodiments, the low dose of ionizing radiation 813 includes a value of between about 3.0 Gy and about 10.0 Gy

In some embodiments the solution 803 has a substantially linear response to the low dose of ionizing radiation 813. For a substantially linear response, the intensity of the color of the solution 817 increases substantially linearly as the low dose of ionizing radiation 813 increases.

The apparatus 801 may further include a detector 819 to analyze the radiated solution 815. In some embodiments, the detector 819 comprises a spectrophotometer. A spectrophotometer is an instrument for measuring electromagnetic radiation in different areas of the electromagnetic spectrum. In some embodiments, the detector 819 includes a human visual system. A human visual system is suitable for use in a variety of color measurement tasks and in particular for identifying changes in color. In some embodiments, the radiated solution 815 has a color and the color has a color intensity that increases with an increase in the low dose of ionizing radiation 813. In come embodiments, the surfactant 809 has a concentration and the solution 803 has a color response and modifying the concentration of the surfactant 809 changes the color response of the solution 803 to the low dose of ionizing radiation 813.

### Composition of Matter

The solution 803 shown in FIG. 8 is a composition of matter. In some embodiments, the solution 803 includes the metallic compound 807, the surfactant 809, and the acid 811. An exemplary metallic compound includes auric chloride (HAuCl₄). An exemplary surfactant includes cetyl trimethylammonium bromide (C₁₆TAB). An exemplary acid suitable for use in forming the solution 803 includes L-ascorbic acid. In some embodiments, the solution 803 is substantially colorless.

### Method of Making the Solution

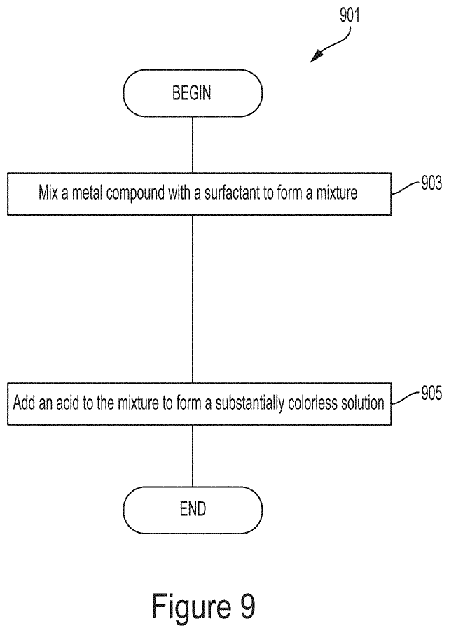

Several methods may be employed to make the solution 803 shown in FIG. 8. FIG. 9 shows a method 901 including mixing a metal compound with a surfactant to form a mixture (block 903) and adding an acid to the mixture to form a substantially colorless solution (block 905). In some embodiments, mixing a metal compound with a surfactant to form a mixture includes mixing auric chloride (HAuCl4) with the surfactant to form the mixture. In some embodiments, adding an acid to the mixture to form a substantially colorless solution includes adding L-ascorbic acid to the mixture to form the substantially colorless solution.

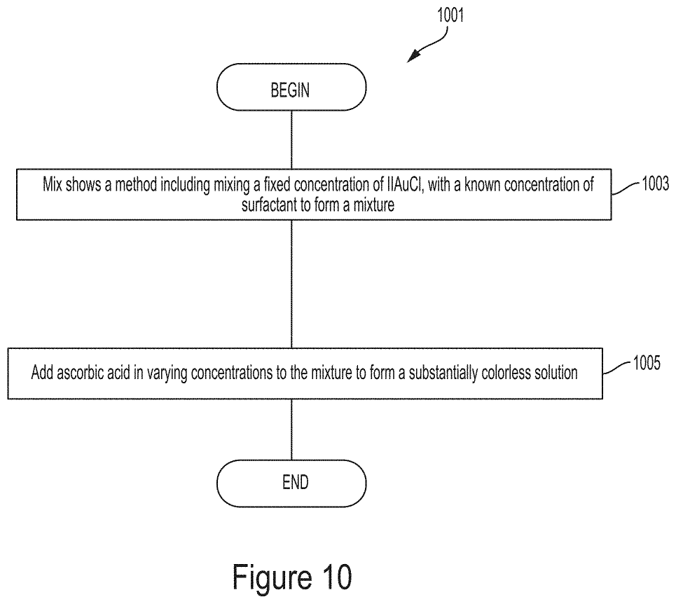

FIG. 10 shows a method 1001 including mixing a fixed concentration of HAuCl₄ with a known concentration of surfactant to form a mixture (block 1003) and adding ascorbic acid in varying concentrations to the mixture to form a substantially colorless solution (block 1005).

### Methods

The apparatus 801 may be employed in a variety of methods useful in detecting radiation.

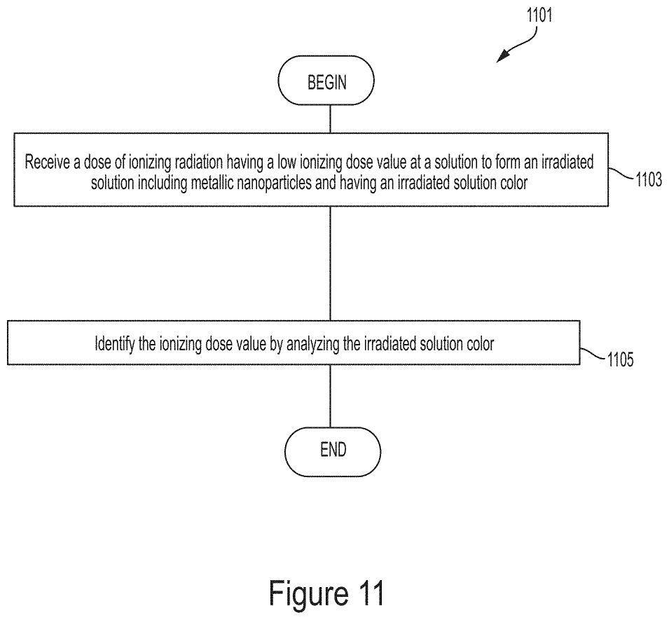

FIG. 11 shows a method 1101 including receiving a dose of ionizing radiation having a low ionizing dose value at a solution to form an irradiated solution including metallic nanoparticles and having an irradiated solution color (block 1103) and identifying the ionizing dose value by analyzing the irradiated solution color (block 1105).

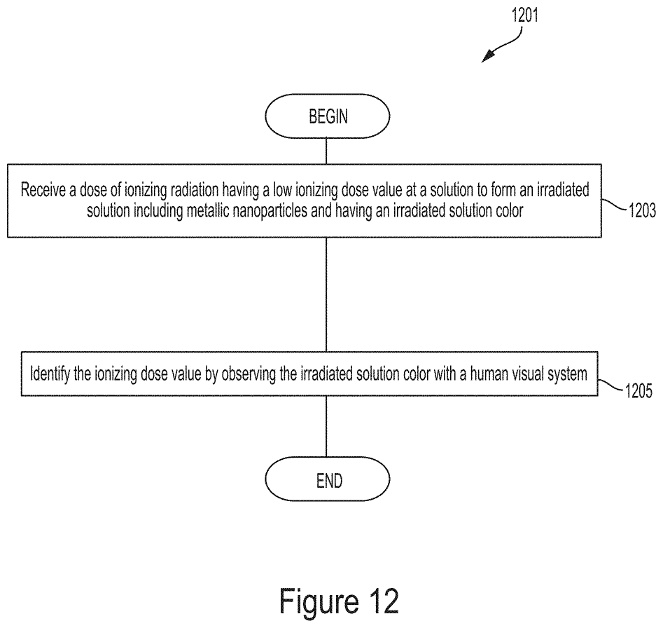

FIG. 12 shows a method 1201 including receiving a dose of ionizing radiation having a low ionizing dose value at a solution to form an irradiated solution including metallic nanoparticles and having an irradiated solution color (block 1203) and identifying the ionizing dose value by observing the irradiated solution color with a human visual system (block 1205).

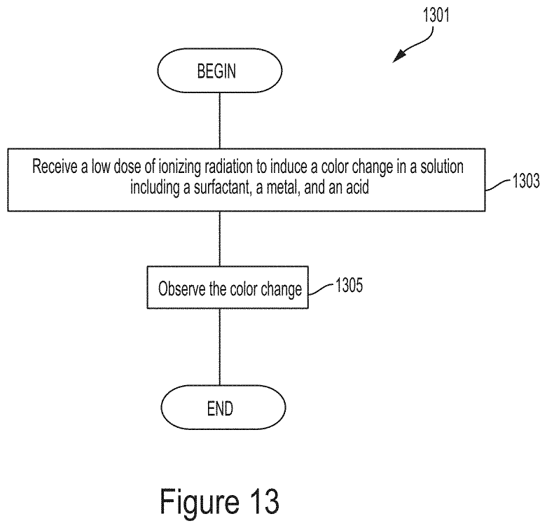

FIG. 13 shows a method 1301 including receiving a low dose of ionizing radiation to induce a color change in a solution including a surfactant, a metal, and an acid (block 1303) and observing the color change (block 13053). In some embodiments, observing the color change comprises observing the color change using a human visual system. In some embodiments, observing the color change includes observing the color change using a spectrophotometer.

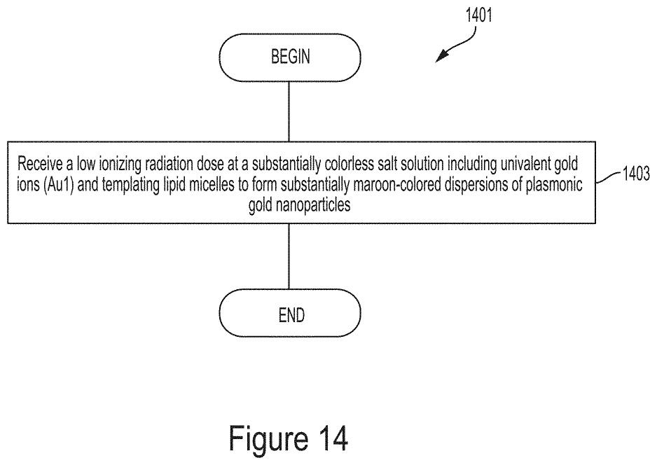

FIG. 14 shows a method 1401 including receiving a low ionizing radiation dose at a substantially colorless salt solution including univalent gold ions (Aul) and templating lipid micelles to form substantially maroon-colored dispersions of plasmonic gold nanoparticles (block 1403).

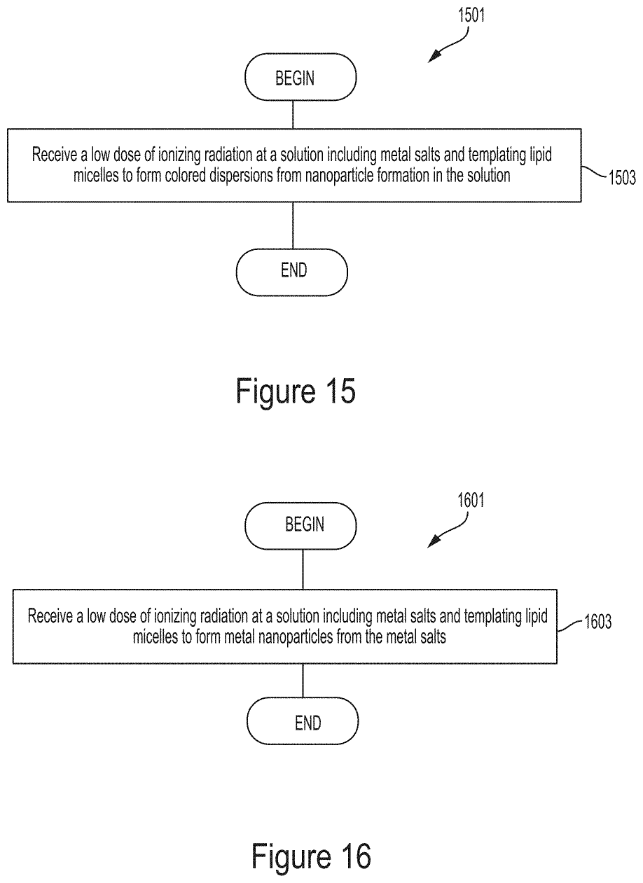

FIG. 15 shows a method 1501 including receiving a low dose of ionizing radiation at a solution including metal salts and templating lipid micelles to form colored dispersions from nanoparticle formations in the solution (block 1503).

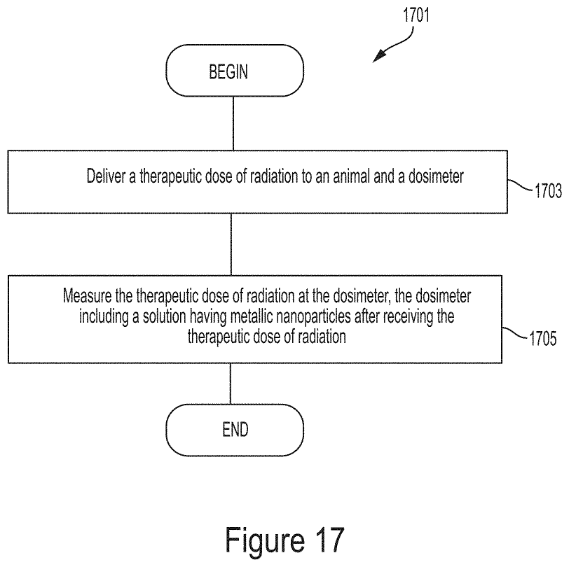

FIG. 16 shows a method 1601 including receiving a low dose of ionizing radiation at a solution including metal salts and templating lipid micelles to form metal nanoparticles from the metal salts (block 1603).

### Therapeutic Methods

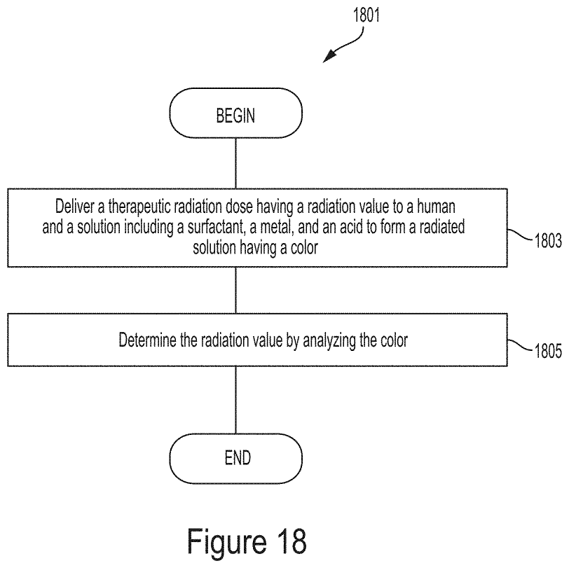

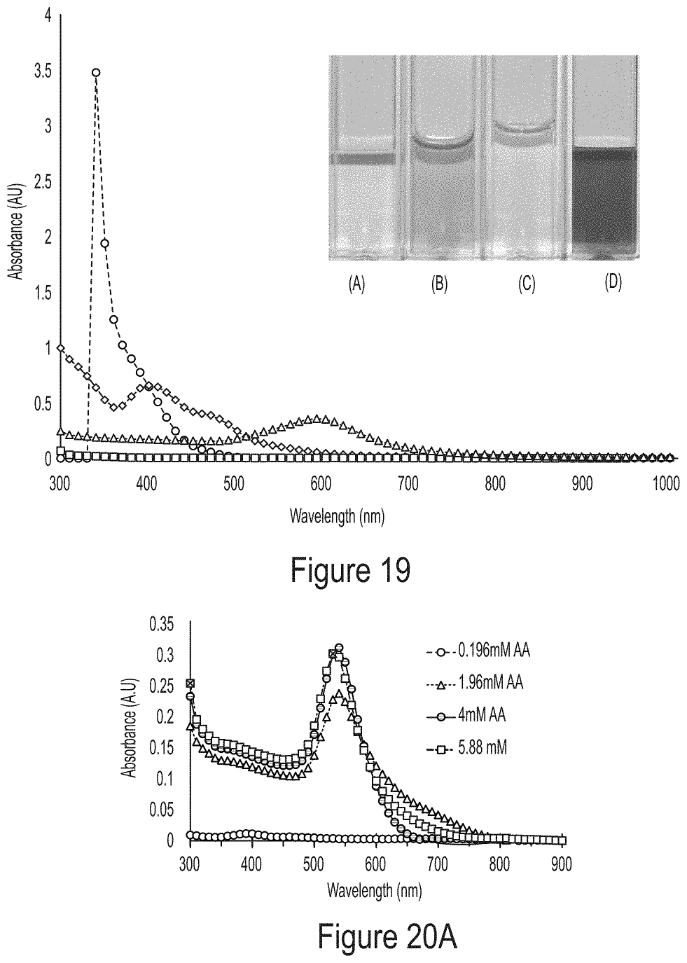

The apparatus 801 shown in FIG. 8 can be employed in a variety of therapeutic methods. For example, FIG. 17 shows a method 1701 that includes delivering a therapeutic dose of radiation to an animal and a dosimeter (block 1703) and measuring the therapeutic dose of radiation at the dosimeter, the dosimeter including a solution having metallic nanoparticles after receiving the therapeutic dose of radiation (block 1705). In another example, FIG. 18 shows a method 1801 that includes delivering a therapeutic radiation dose having a radiation value to a human and a solution including a surfactant, a metal, and an acid to form a radiated solution having a color (block 1803) and determining the radiation value by analyzing the color (block 1805).

### EXPERIMENTAL

Materials: Gold(III) chloride trihydrate (HAuCl₄.3H₂O), trimethyloctylammonium bromide (C₈TAB) (>98%), dodecyltrimethylammonium bromide (C₁₂TAB) (≥98%) and L-Ascorbic acid (AA) were purchased from Sigma-Aldrich. Cetyl trimethylammonium bromide (C₁₆TAB) was purchased from MP chemicals. All chemicals were used as received from the manufacturer without any additional purification.

Sample preparation for irradiation: First, 30 μL of 0.01 M HAuCl₄ were mixed with 600 μL of 0.05 M Cₓ₌8,12,16TAB. Upon addition of 30 μL (0.196 mM), 300 μL (1.96 mM), 600 μL (3.92 mM approximated as 4 mM) and 900 μL (5.88 mM) of 0.01 M L-Ascorbic acid, the solution turned colorless after shaking; the concentrations of ascorbic acid were thus varied in order to examine its effect on nanoparticle formation (FIGS. 20A-20B, Supporting Information section). Unless specifically mentioned, the volume of AA used is 900 μL. The measured pH of the solution was between 2.9 and 3.1. Samples were prepared at Banner-MD Anderson Cancer Center, Gilbert, Ariz. prior to radiation.

Radiation Conditions: A TrueBeam linear accelerator was used to irradiate the samples. Samples were radiated at a dose rate of (15.6 Gy/min). The samples containing surfactant at a concentration of 20 mM and 10 mM were radiated at doses of 0 (Control), 1.1, 3.2, 5.3, 10.5, 15.8, 26.3, 36.9 and 47.4 Gy. These are reported as 0, 1, 3, 5, 10, 16, 26, 37 and 47 Gy respectively in the article. The samples containing surfactant at a concentration 2 mM and 4 mM were irradiated with 0 (Control), 0.5, 1, 1.5, 2, 2.5, 3, 5, 7.5, 10, 12.5 and 15 Gy. After irradiation the samples were transported back to Arizona State University in Tempe, Ariz. (one-way travel time of approximately 30 minutes).

Absorbance Spectroscopy: Absorbance profiles of the radiated and the control samples were measured using a BioTek Synergy 2 plate reader. Absorbance values from 150 μL of sample were measured from 300 to 900 nm with a step size of 10 nm in a 96 well plate. Nanopure water (18.2 MΩcm) was used as a blank in all cases. The absorbance was corrected for offset by subtracting A₉₀₀ nm and the presence of a peak between 500 and 700 nm was used as an indicator for gold nanoparticle formation.

Determination of Critical Micellar Concentration (CMC): Pyrene (60 μL of 2×10⁻⁵M) in acetone was added to 20 ml glass vials. Upon acetone evaporation, 2 ml of C₁₆TAB of varying concentrations was added and stirred for 6 hours at room temperature. To achieve the similar conditions as the irradiation experiments, 30 μL of 10 mM gold salt+600 μL of the above prepared C₁₆TAB+900 μL of 10 mM ascorbic acid were mixed. A fluorescence spectrophotometer with an excitation scan range of 300-360 nm and an emission wavelength of 390 nm was used. Ratio of I₃₃₇/I₃₃₄ determined as a function of the surfactant concentration was used to calculate the CMC using pyrene as the probe based on methods described in the literature.

Dynamic Light Scattering (DLS) Measurements: 50 μL of the sample was transferred into a cuvette and placed into a Zetasizer Nano instrument. The software was set up to carry out measurements with autocorrelation. Thereafter, the average diameter along with the polydispersity index (PDI) were recorded based on the software readout.

Transmission Electron Microscopy (TEM): Samples for TEM were prepared by casting a drop of the solution onto a carbon film on a copper mesh grid. The samples were then dried in air. The above process was repeated several times to ensure good coverage. Dried samples were visualized using a CM200-FEG instrument operating at 200 kV.

TABLE 1
Absorbance values measured 7 hours following exposure of endorectal balloons
with the plasmonic nanosensor (20 mM C₁₆TAB concentration) following
exposure to different doses of ionizing radiation. The calibration equation
used was Absorbance = 0.0092*Dose - 0.0356. The 0 Gy data point
is outside the linear range (5-37 Gy) of the nanosensor, and the nanosensor
is able to detect X-ray radiation in the linear range.

| | | Calculated Dose from | Average Radiation |
|---|---|---|---|
| Delivered Dose | Measured Absorbance | the calibration curve | Dose Delivered ± S.D |
| (Gy) | (A.U) | (Gy) | (Gy) |
| 0 | 0.003, 0.002, 0.009 | 4.19, 4.09, 4.85 | 4.38 ± 0.41 |
| 7.9 | 0.05, 0.015, 0.045 | 9.30, 5.50, 8.76 | 7.85 ± 2.05 |
| 10.5 | 0.061, 0.035, 0.032 | 10.50, 7.67, 7.35 | 8.51 ± 1.73 |

TABLE 2
X-ray Radiation dose determined using the plasmonic nanosensor placed on 10 an
endorectal balloon in a prostate phantom as shown in FIG. 8. The absorbance was
determined 2 h after radiation exposure using the equation Absorbance = 0.1597*Dose
- 0.0542. 0.5 Gy to 1.5 Gy was the dose range used for determining the calibration curve.
A C₁₆TAB concentration of 2 mM was used in these studies.

| Capsule | | | |
|---|---|---|---|
| No. (Actual | | Calculated Dose from | Average Radiation |
| Dose Delivered | Measured Absorbance | the calibration curve | Dose Delivered ± S.D |
| in Gy) | (A.U) | (Gy) | (Gy) |
| 1 (1) | 0.12, 0.138, 0.154 | 1.09, 1.20, 1.30 | 1.20 ± 0.11 |
| 2 (1) | 0.105, 0.154, 0.137 | 1.00, 1.30, 1.20 | 1.17 ± 0.16 |
| 3 (0.5) | 0.016, 0.03, 0.025 | 0.44, 0.53, 0.50 | 0.49 ± 0.04 |

TABLE 3
Average hydrodynamic diameters of gold nanoparticles formed after
irradiation along with their corresponding polydispersity indices.

| | | | | | Average |
|---|---|---|---|---|---|
| | | | Average | STD DEV | Polydispersity |
| | | | Diameter | Diameter | Index |
| Surfactant | Dose | | (nm) | (nm) | (PDI) |
| C₁₆ 20 mM | 1 | Gy | 138.4 | 5.3 | 0.2 |
| | 3 | Gy | 122.8 | 1.9 | 0.2 |
| | 5 | Gy | 121.1 | 20.7 | 0.3 |
| | 10 | Gy | 102.3 | 13.2 | 0.2 |
| | 16 | Gy | 88.5 | 12.1 | 0.2 |
| | 26 | Gy | 72.6 | 4.7 | 0.2 |
| | 37 | Gy | 57.3 | 4.0 | 0.3 |
| | 47 | Gy | 45.5 | 3.4 | 0.3 |
| C₁₆ 2 mM | 0.5 | Gy | 81.9 | 8.9 | 0.3 |
| | 1 | Gy | 60.2 | 6.1 | 0.3 |
| | 1.5 | Gy | 48.2 | 7.3 | 0.4 |
| | 2 | Gy | 42.9 | 3.8 | 0.4 |
| | 2.5 | Gy | 39.8 | 3.6 | 0.4 |
| C₁₆ 4 mM | 1 | Gy | 133.4 | 10.4 | 0.2 |
| | 3 | Gy | 124.2 | 5.2 | 0.2 |
| | 5 | Gy | 105.3 | 6.3 | 0.2 |
| | 7.5 | Gy | 88.6 | 8.1 | 0.3 |
| | 10 | Gy | 92.6 | 8.6 | 0.3 |
| | 12.5 | Gy | 81.3 | 6.9 | 0.3 |
| | 15 | Gy | 74.2 | 5.5 | 0.3 |
| | 26 | Gy | 57.4 | 2.4 | 0.3 |
| | 37 | Gy | 32.0 | 0.4 | 0.5 |
| | 47 | Gy | 22.1 | 1.3 | 0.6 |
| C₁₆ 10 mM | 1 | Gy | 126.4 | 1.5 | 0.2 |
| | 3 | Gy | 127.1 | 1.6 | 0.2 |
| | 5 | Gy | 124.8 | 2.1 | 0.2 |
| | 10 | Gy | 124.9 | 5.0 | 0.2 |
| | 16 | Gy | 106.2 | 5.4 | 0.2 |
| | 26 | Gy | 72.2 | 7.1 | 0.2 |
| | 37 | Gy | 59.4 | 3.3 | 0.3 |
| | 47 | Gy | 50.9 | 2.3 | 0.2 |
| C₁₂ 20 mM | 1 | Gy | 141.6 | 32.2 | 0.5 |
| | 3 | Gy | 112.2 | 5.3 | 0.2 |
| | 5 | Gy | 75.2 | 5.0 | 0.3 |
| | 10 | Gy | 40.4 | 1.0 | 0.5 |
| | 16 | Gy | 23.9 | 1.1 | 0.6 |
| | 26 | Gy | 15.7 | 0.8 | 0.6 |
| | 37 | Gy | 17.9 | 0.7 | 0.6 |
| | 47 | Gy | 21.6 | 2.7 | 0.6 |

TABLE 4
A list of surfactants which could be potentially be used as an alternative to the current
surfactants. Any derivative of the above compounds could also be potentially be used.

| | | Molecular |
|---|---|---|
| Surfactant Name | Structure | Formula |
| Acetylcholinechloride ≥ 99% (TLC) | 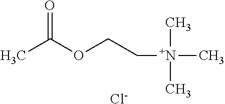 | C₇H₁₆ClNO₂ |
| Aliquat ® 336 | | |
| (2-Aminoethyl)trimethylammonium chloride hydrochloride 99% | 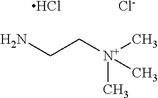 | C₅H₁₅ClN₂•HCl |
| Arquad ® 2HT-75 | | |
| Benzalkonium chloride ≥ 95.0% (T) | 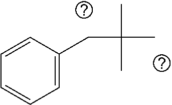 | |
| Benzalkonium chloride | 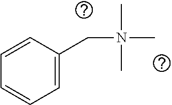 | |
| Benzalkonium chloride solution PharmaGrade. | 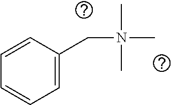 | |
| Benzalkonium chloride solution ≥ 50% (via Cl) 50% in H₂O | 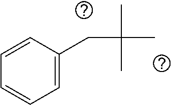 | |
| Benzyldimethyldecylammonium chloride ≥ 97.0% (AT) | 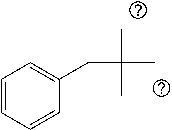 | C₁₉H₃₄ClN |
| Benzyldimethyldodecylammonium chloride ≥ 99.0% (AT) | 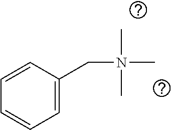 | C₂₁H₃₈ClN |
| Benzyldimethylhexadecylammonium chloride ≥ 97.0% (dried material, AT) | 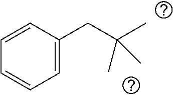 | C₂₅H₄₆ClN |
| Benzyldimethylhexylammonium chloride ≥ 96.0% (AT) | 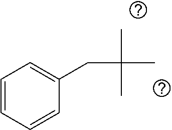 | C₁₅H₂₆ClN |
| Benzyldimethyl(2-hydroxyethyl)ammonium chloride ≥ 97.0% (AT) | 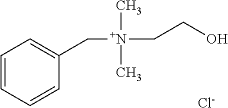 | C₁₁H₁₈ClNO |
| Benzyldimethyloctylammonium chloride ≥ 96.0% (AT) | 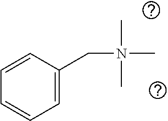 | C₁₇H₃₀ClN |
| Benzyldimethyltetradecylammonium chloride anhydrous, ≥99.0% (AT) | 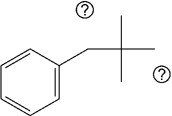 | C₂₃H₄₂ClN |
| Benzyldimethyltetradecylammonium chloride dihydrate 98% | 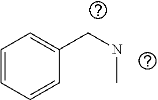 | C₂₃H₄₂ClN•2H₂O |
| Benzyldodecyldimethylammonium bromide ≥ 99.0% (AT) | 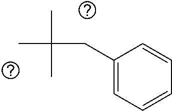 | C₂₁H₃₈BrN |
| Benzyldodecyldimethylammonium bromide purum, ≥97.0% (AT) | 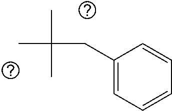 | C₂₁H₃₈BrN |
| Benzyltributylammonium bromide ≥ 99.0% | 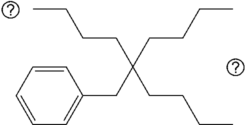 [SMILES: CCCCC(CCCC)(CCCC)Cc1ccccc1] | C₁₉H₃₄BrN |
| Benzyltributylammonium chloride ≥ 98% | 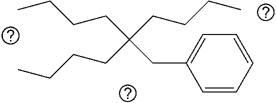 [SMILES: CCCCC(CCCC)(CCCC)Cc1ccccc1] | C₁₉H₃₄ClN |
| Benzyltributylammonium iodide 97% | 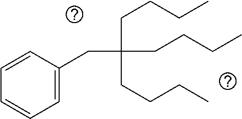 [SMILES: CCCCC(CCCC)(CCCC)Cc1ccccc1] | C₁₉H₃₄IN |
| Benzyltriethylammonium bromide 99% | 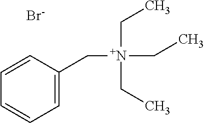 | C₁₃H₂₂BrN |
| Benzyltriethylammonium chloride 99% | 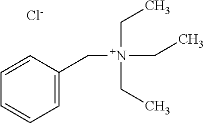 | C₁₃H₂₂ClN |
| Benzyltriethylammonium chloride monohydrate 97% | 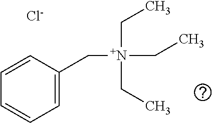 | C₁₃H₂₂ClN•H₂O |
| Benzyltrimethylammonium bromide 97% | 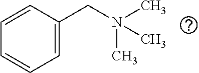 | C₁₀H₁₆BrN |
| Benzyltrimethylammonium chloride purum, ≥98.0% (AT) | 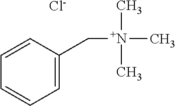 | C₁₀H₁₆ClN |
| Benzyltrimethylammonium chloride 97% | 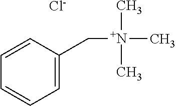 | C₁₀H₁₆ClN |
| Benzyltrimethylammonium chloride solution technical, ~60% in H₂O | 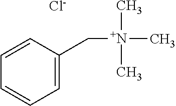 | C₁₀H₁₆ClN |
| Benzyltrimethylammonium dichloroiodate 97% | 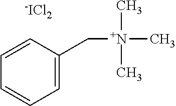 | C₁₀H₁₆Cl₂IN |
| Benzyltrimethylammonium tetrachloroiodate ≥ 98.0% (AT) | 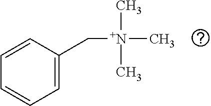 | C₁₀H₁₆Cl₄IN |
| Benzyltrimethylammonium tribromide purum, ≥97.0% (AT) | 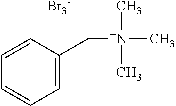 | C₁₀H₁₆Br₃N |
| Benzyltrimethylammonium tribromide 97% | 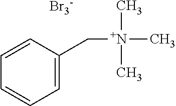 | C₁₀H₁₆Br₃N |
| Bis(triphenylphosphoranylidene)ammonium chloride 97% | 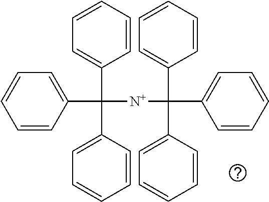 [SMILES: c1ccc(C([NH2+]C(c2ccccc2)(c2ccccc2)c2ccccc2)(c2ccccc2)c2ccccc2)cc1] | C₃₆H₃₀ClNP₂ |
| Boc-D-Lys(2-Cl—Z)—OH ≥ 98.0% (TLC) | 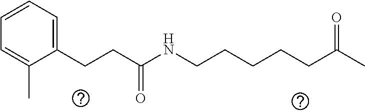 [SMILES: CC(=O)CCCCCNC(=O)CCc1ccccc1C] | C₁₉H₂₇ClN₂O₆ |
| (2-Bromoethyl)trimethylammonium bromide 98% | 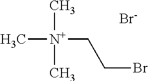 | C₅H₁₃Br₂N |
| (5-Bromopentyl)trimethylammonium bromide 97% | 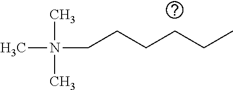 | C₈H₁₉Br₂N |
| (3-Bromopropyl)trimethylammonium bromide 97% | 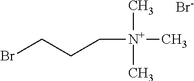 | C₆H₁₅Br₂N |
| S-Butyrylthiocholine iodide puriss., ≥99.0% (AT) | 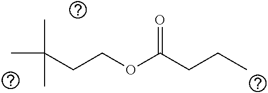 | C₉H₂₀INOS |
| Carbamoylcholine chloride 99% | 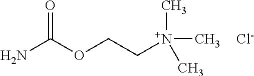 | C₆H₁₅ClN₂O₂ |
| (3-Carboxypropyl)trimethylammonium chloride technical grade | 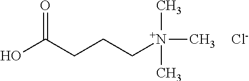 | C₇H₁₆ClNO₂ |
| Cetyltrimethylammonium chloride solution 25 wt. % in H₂O | 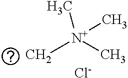 | C₁₉H₄₂ClN |
| Cetyltrimethylammonium hydrogensulfate 99% | 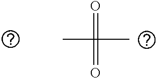 | C₁₉H₄₃NO₄S |
| (2-Chloroethyl)trimethylammonium chloride 98% | 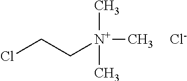 | C₅H₁₃Cl₂N |
| (3-Chloro-2-hydroxypropyl)trimethylammonium chloride solution purum, ~65% in H₂O (T) | 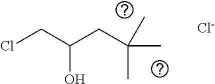 | C₆H₁₅Cl₂NO |
| (3-Chloro-2-hydroxypropyl)trimethylammonium chloride solution 60 wt. % in H₂O | 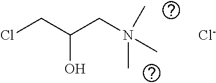 | C₆H₁₅Cl₂NO |
| Choline chloride ≥ 99% | 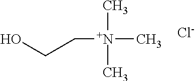 | C₅H₁₄ClNO |
| Decyltrimethylammonium bromide ≥ 98.0% (NT) | 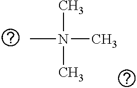 | C₁₃H₃₀BrN |
| Diallyldimethylammonium chloride ≥ 97.0% (AT) | 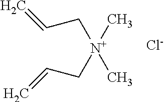 | C₈H₁₆ClN |
| Diallyldimethylammonium chloride solution 65 wt. % in H₂O | 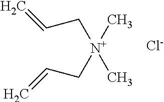 | C₈H₁₆ClN |
| Didecyldimethylammonium bromide 98% | 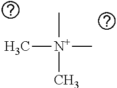 | C₂₂H₄₈BrN |
| Didodecyldimethylammonium bromide 98% | 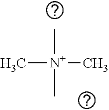 | C₂₆H₅₆BrN |
| Dihexadecyldimethylammonium bromide 97% | 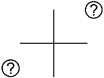 | C₃₄H₇₂BrN |
| Dimethyldioctadecylammonium bromide ≥ 98.0% (AT) | 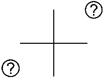 | C₃₈H₈₀BrN |
| Dimethyldioctadecylammonium chloride ≥ 97.0% (AT) | 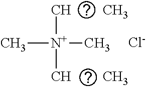 | C₃₈H₈₀ClN |
| Dimethylditetradecylammonium bromide ≥ 97.0% (NT) | 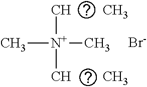 | C₃₀H₆₄BrN |
| Dimethyloctadecyl[3-(trimethoxysilyl)propyl]ammonium chloride solution 42 wt. % in methanol | 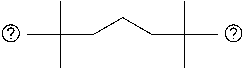 | C₂₆H₅₈ClNO₃Si |
| Dodecylethyldimethylammonium bromide ≥ 98.0% (AT) | 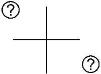 | C₁₆H₃₆BrN |
| Dodecyltrimethylammonium chloride ≥ 99.0% (AT) | 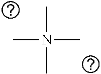 | C₁₅H₃₄ClN |
| Dodecyltrimethylammonium chloride purum, ≥98.0% (AT) | 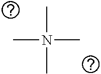 | C₁₅H₃₄ClN |
| Domiphen bromide 97% | 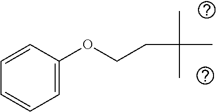 | C₂₂H₄₀BrNO |
| Ethyltrimethylammonium iodide ≥ 99.0% | 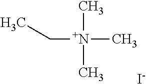 | C₅H₁₄IN |
| Girard's reagent T 99% | 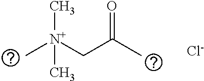 | C₅H₁₄ClN₃O |
| Glycidyltrimethylammonium chloride technical, ≥90% (calc. based on dry substance, AT) | 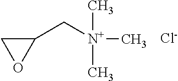 | C₆H₁₄ClNO |
| Heptadecafluorooctanesulfonic acid tetraethylammonium salt purum, ≥98.0% (T) | 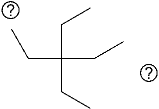 | C₁₆H₂₀F₁₇NO₃S |
| Heptadecafluorooctanesulfonic acid tetraethylammonium salt 98% | 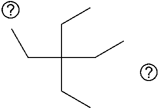 | C₁₆H₂₀F₁₇NO₃S |
| Hexadecyl(2-hydroxyethyl)dimethylammonium dihydrogen phosphate solution ~30% in H₂O | 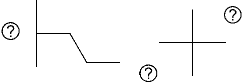 | C₂₀H₄₆NO₅P |
| Hexadecyltrimethylammonium bisulfate purum, ≥97.0% (T) | 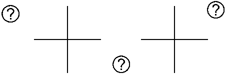 | C₁₉H₄₃NO₄S |
| Hexadecyltrimethylammonium bromide ≥ 96.0% (AT) | 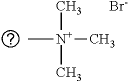 | C₁₉H₄₂BrN |
| Hexadecyltrimethylammonium chloride ≥ 98.0% (NT) | 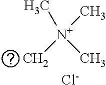 | C₁₉H₄₂ClN |
| Hexadecyltrimethylammonium chloride solution purum, ~25% in H₂O | 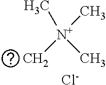 | C₁₉H₄₂ClN |
| Hexamethonium bromide ≥ 95.0% (AT) | 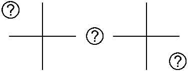 | C₁₂H₃₀Br₂N₂ |
| Hexyltrimethylammonium bromide ≥ 98.0% (AT) | 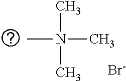 | C₉H₂₂BrN |
| Hyamine ® 1622 solution 4 mM in H₂O | 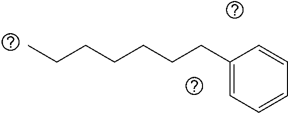 | |
| Malondialdehyde tetrabutylammonium salt ≥ 96.0% (NT) | 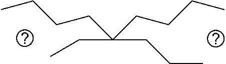 | C₁₉H₃₉NO₂ |
| Methyltrioctylammonium bromide 97% | 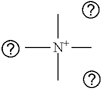 | C₂₅H₅₄BrN |
| Methyltrioctylammonium chloride ≥ 97.0% (AT) | 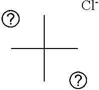 | C₂₅H₅₄ClN |
| Methyltrioctylammonium hydrogen sulfate ≥ 95.0% (T) | 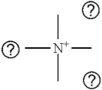 | C₂₅H₅₅NO₄S |
| Methyltrioctylammonium thiosalicylate ≥ 95% (C) | 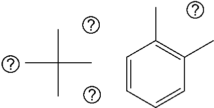 | C₃₂H₅₉NO₂S |
| Myristyltrimethylammonium bromide 98% (AT) | 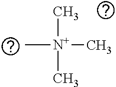 | C₁₇H₃₈BrN |
| (4-Nitrobenzyl)trimethylammonium chloride 98% | 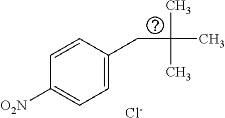 | C₁₀H₁₅ClN₂O₂ |
| OXONE ® tetrabutylammonium salt technical, ~1.6% active oxygen basis | 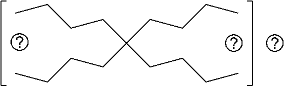 | |
| Tetrabutylammonium acetate for electrochemical analysis, ≥99.0% | 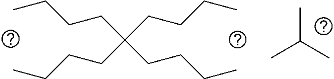 | C₁₈H₃₉NO₂ |
| Tetrabutylammonium acetate 97% | 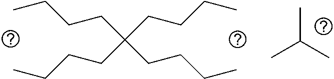 | C₁₈H₃₉NO₂ |
| Tetrabutylammonium acetate technical, ≥90% (T) | 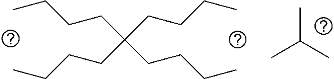 | C₁₈H₃₉NO₂ |
| Tetrabutylammonium acetate solution 1.0M in H₂O | 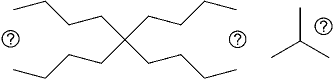 | C₁₈H₃₉NO₂ |
| Tetrabutylammonium benzoate for electrochemical analysis, ≥99.0% | 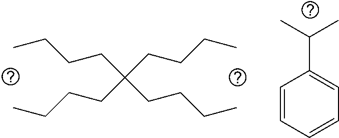 | C₂₃H₄₁NO₂ |
| Tetrabutylammonium bisulfate puriss., ≥99.0% (T) | 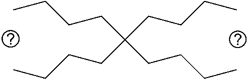 [SMILES: CCCCC(CCCC)(CCCC)CCCC] | C₁₆H₃₇NO₄S |
| Tetrabutylammonium bisulfate purum, ≥97.0% (T) | 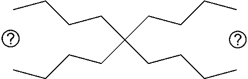 [SMILES: CCCCC(CCCC)(CCCC)CCCC] | C₁₆H₃₇NO₄S |
| Tetrabutylammonium bisulfate solution ~55% in H₂O | 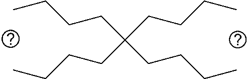 [SMILES: CCCCC(CCCC)(CCCC)CCCC] | C₁₆H₃₇NO₄S |
| Tetrabutylammonium bromide ACS reagent, ≥98.0% | 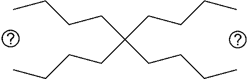 [SMILES: CCCCC(CCCC)(CCCC)CCCC] | C₁₆H₃₆BrN |
| Tetrabutylammonium bromide ReagentPlus ®, ≥99.0% | 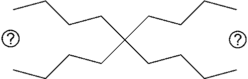 [SMILES: CCCCC(CCCC)(CCCC)CCCC] | C₁₆H₃₆BrN |
| Tetrabutylammonium bromide solution 50 wt. % in H₂O | 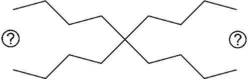 [SMILES: CCCCC(CCCC)(CCCC)CCCC] | C₁₆H₃₆BrN |
| Tetrabutylammonium chloride ≥ 97.0% (NT) | 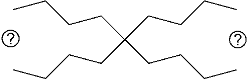 [SMILES: CCCCC(CCCC)(CCCC)CCCC] | C₁₆H₃₆ClN |
| Tetrabutylammonium chloride hydrate 98% | 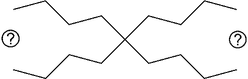 [SMILES: CCCCC(CCCC)(CCCC)CCCC] | C₁₆H₃₆ClN |
| Tetrabutylammonium cyanate technical | 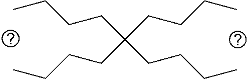 [SMILES: CCCCC(CCCC)(CCCC)CCCC] | C₁₇H₃₆N₂O |
| Tetrabutylammonium cyanide purum, ≥95.0% (AT) | 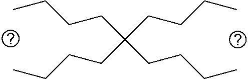 [SMILES: CCCCC(CCCC)(CCCC)CCCC] | C₁₇H₃₆N₂ |
| Tetrabutylammonium cyanide 95% | 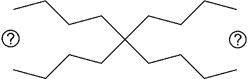 [SMILES: CCCCC(CCCC)(CCCC)CCCC] | C₁₇H₃₆N₂ |
| Tetrabutylammonium cyanide technical, ≥80% | 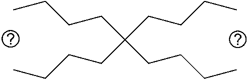 [SMILES: CCCCC(CCCC)(CCCC)CCCC] | C₁₇H₃₆N₂ |
| Tetrabutylammonium difluorotriphenylsilicate 97% | 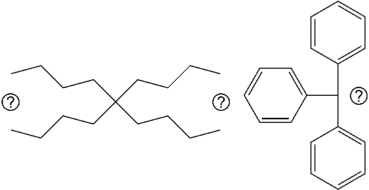 | C₃₄H₅₁F₂NSi |
| Tetrabutylammonium difluorotriphenylstannate 97% | 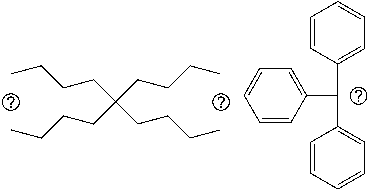 | C₃₄H₅₁F₂NSn |
| Tetrabutylammonium glutaconaldehyde enolate hydrate ≥ 97.0% (T) | 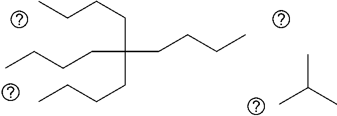 | C₂₁H₄₁NO₂•xH₂O |
| Tetrabutylammonium heptadecafluorooctanesulfonate ≥ 95.0% (H-NMR) | 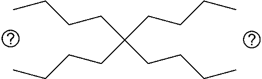 | C₂₄H₃₆F₁₇NO₃S |
| Tetrabutylammonium hexafluorophosphate for electrochemical analysis, ≥99.0% | 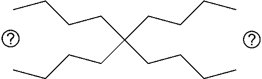 [SMILES: CCCCC(CCCC)(CCCC)CCCC] | C₁₆H₃₆F₆NP |
| Tetrabutylammonium hexafluorophosphate purum, ≥98.0% (CHN) | 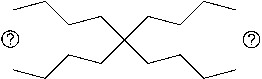 [SMILES: CCCCC(CCCC)(CCCC)CCCC] | C₁₆H₃₆F₆NP |
| Tetrabutylammonium hexafluorophosphate 98% | 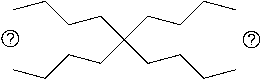 [SMILES: CCCCC(CCCC)(CCCC)CCCC] | C₁₆H₃₆F₆NP |
| Tetrabutylammonium hydrogen difluoride solution technical, ~50% in methylene chloride (T) | 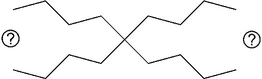 [SMILES: CCCCC(CCCC)(CCCC)CCCC] | C₁₆H₃₇F₂N |
| Tetrabutylammonium hydrogen difluoride solution ~50% in acetonitrile | 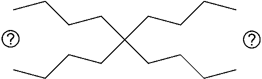 [SMILES: CCCCC(CCCC)(CCCC)CCCC] | C₁₆H₃₇F₂N |
| Tetrabutylammonium hydrogensulfate anhydrous, free-flowing, Redi-Dri ™, 97% | 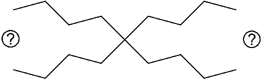 [SMILES: CCCCC(CCCC)(CCCC)CCCC] | C₁₆H₃₇NO₄S |
| Tetrabutylammonium hydrogensulfate 97% | 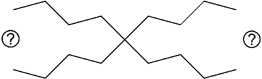 [SMILES: CCCCC(CCCC)(CCCC)CCCC] | C₁₆H₃₇NO₄S |
| Tetrabutylammonium iodide for electrochemical analysis, ≥99.0% | 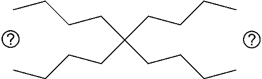 [SMILES: CCCCC(CCCC)(CCCC)CCCC] | C₁₆H₃₆IN |
| Tetrabutylammonium iodide ≥ 99.0% (AT) | 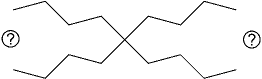 [SMILES: CCCCC(CCCC)(CCCC)CCCC] | C₁₆H₃₆IN |
| Tetrabutylammonium iodide reagent grade, 98% | 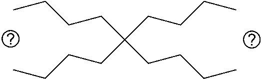 [SMILES: CCCCC(CCCC)(CCCC)CCCC] | C₁₆H₃₆IN |
| Tetrabutylammonium methanesulfonate ≥ 97.0% (T) | 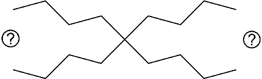 [SMILES: CCCCC(CCCC)(CCCC)CCCC] | C₁₇H₃₉NO₃S |
| Tetrabutylammonium methoxide solution 20% in methanol (NT) | 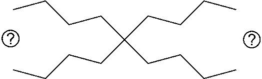 [SMILES: CCCCC(CCCC)(CCCC)CCCC] | C₁₇H₃₉NO |
| Tetrabutylammonium nitrate purum, ≥97.0% (NT) | 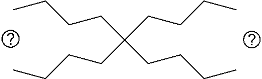 [SMILES: CCCCC(CCCC)(CCCC)CCCC] | C₁₆H₃₆N₂O₃ |
| Tetrabutylammonium nitrate 97% | 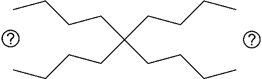 [SMILES: CCCCC(CCCC)(CCCC)CCCC] | C₁₆H₃₆N₂O₃ |
| Tetrabutylammonium nitrite ≥ 97.0% (NT) | 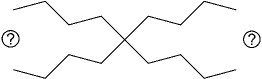 [SMILES: CCCCC(CCCC)(CCCC)CCCC] | C₁₆H₃₆N₂O₂ |
| Tetrabutylammonium nonafluorobutanesulfonate ≥ 98.0% | 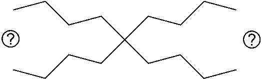 [SMILES: CCCCC(CCCC)(CCCC)CCCC] | C₂₀H₃₆F₉NO₃S |
| Tetrabutylammonium perchlorate for electrochemical analysis, ≥99.0% | 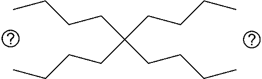 [SMILES: CCCCC(CCCC)(CCCC)CCCC] | C₁₆H₃₆ClNO₄ |
| Tetrabutylammonium perchlorate ≥ 98.0% (T) | 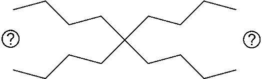 [SMILES: CCCCC(CCCC)(CCCC)CCCC] | C₁₆H₃₆ClNO₄ |
| Tetrabutylammonium phosphate monobasic puriss., ≥99.0% (T) | 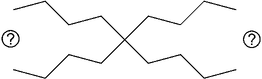 [SMILES: CCCCC(CCCC)(CCCC)CCCC] | C₁₆H₃₈NO₄P |
| Tetrabutylammonium phosphate monobasic solution 1.0M in H₂O | 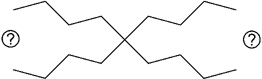 [SMILES: CCCCC(CCCC)(CCCC)CCCC] | C₁₆H₃₈NO₄P |
| Tetrabutylammonium phosphate monobasic solution puriss., ~1M in H₂O | 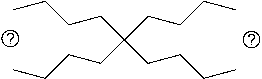 [SMILES: CCCCC(CCCC)(CCCC)CCCC] | C₁₆H₃₈NO₄P |
| Tetrabutylammonium succinimide ≥ 97.0% (NT) | 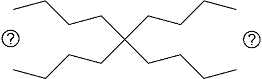 [SMILES: CCCCC(CCCC)(CCCC)CCCC] | C₂₀H₄₀N₂O₂ |
| Tetrabutylammonium sulfate solution 50 wt. % in H₂O | 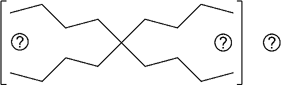 | C₃₂H₇₂N₂O₄S |
| Tetrabutylammonium tetrabutylborate 97% | 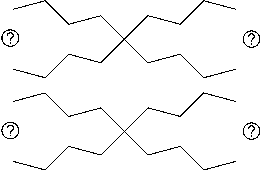 | C₃₂H₇₂BN |
| Tetrabutylammonium tetrafluoroborate for electrochemical analysis, ≥99.0% | 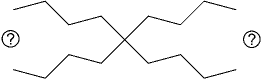 [SMILES: CCCCC(CCCC)(CCCC)CCCC] | C₁₆H₃₆BF₄N |
| Tetrabutylammonium tetrafluoroborate puriss., ≥99.0% (T) | 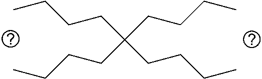 [SMILES: CCCCC(CCCC)(CCCC)CCCC] | C₁₆H₃₆BF₄N |
| Tetrabutylammonium tetrafluoroborate 99% | 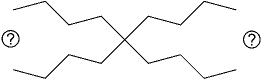 [SMILES: CCCCC(CCCC)(CCCC)CCCC] | C₁₆H₃₆BF₄N |
| Tetrabutylammonium tetraphenylborate for electrochemical analysis, ≥99.0% | 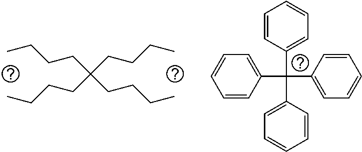 | C₄₀H₅₆BN |
| Tetrabutylammonium tetraphenylborate puriss., ≥99.0% (NT) | 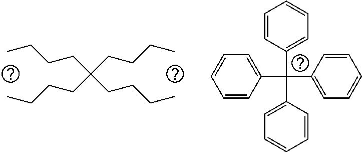 | C₄₀H₅₆BN |
| Tetrabutylammonium tetraphenylborate 99% | 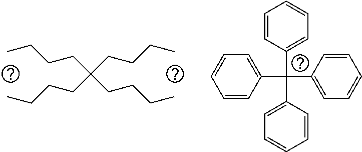 | C₄₀H₅₆BN |
| Tetrabutylammonium thiocyanate purum, ≥99.0% (AT) | 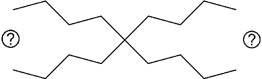 [SMILES: CCCCC(CCCC)(CCCC)CCCC] | C₁₇H₃₆N₂S |
| Tetrabutylammonium thiocyanate 98% | 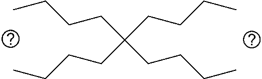 [SMILES: CCCCC(CCCC)(CCCC)CCCC] | C₁₇H₃₆N₂S |
| Tetrabutylammonium p-toluenesulfonate purum, ≥99.0% (T) | 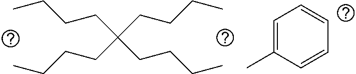 | C₂₃H₄₃NO₃S |
| Tetrabutylammonium p-toluenesulfonate 99% | 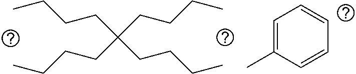 | C₂₃H₄₃NO₃S |
| Tetrabutylammonium tribromide purum, ≥98.0% (RT) | 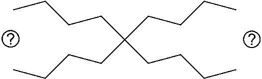 [SMILES: CCCCC(CCCC)(CCCC)CCCC] | C₁₆H₃₆Br₃N |
| Tetrabutylammonium tribromide 98% | 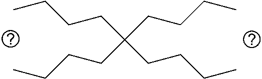 [SMILES: CCCCC(CCCC)(CCCC)CCCC] | C₁₆H₃₆Br₃N |
| Tetrabutylammonium trifluoromethanesulfonate ≥ 99.0% (T) | 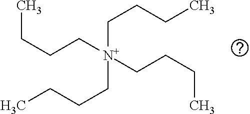 [SMILES: CCCC[N+](CCCC)(CCCC)CCCC] | C₁₇H₃₆F₃NO₃S |
| Tetrabutylammonium triiodide ≥ 97.0% (AT) | 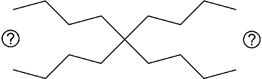 [SMILES: CCCCC(CCCC)(CCCC)CCCC] | C₁₆H₃₆I₃N |
| Tetradodecylammonium bromide ≥ 99.0% (AT) | 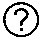 | C₄₈H₁₀₀BrN |
| Tetradodecylammonium chloride ≥ 97.0% (AT) | 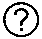 | C₄₈H₁₀₀ClN |
| Tetraethylammonium acetate tetrahydrate 99% | 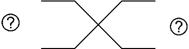 | C₁₀H₂₃NO₂•4H₂O |
| Tetraethylammonium benzoate for electrochemical analysis, ≥99.0% | 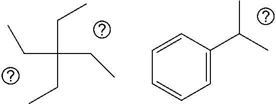 | C₁₅H₂₅NO₂ |
| Tetraethylammonium bicarbonate ≥ 95.0% (T) | 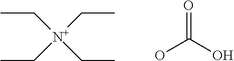 | C₉H₂₁NO₃ |
| Tetraethylammonium bistrifluoromethanesulfonimidate for electronic purposes, ≥99.0% | 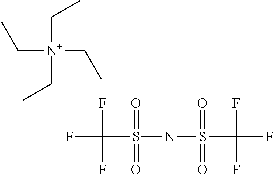 | C₁₀H₂₀F₆N₂O₄S₂ |
| Tetraethylammonium bromide ReagentPlus ®, 99% | 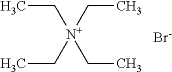 | C₈H₂₀BrN |
| Tetraethylammonium bromide reagent grade, 98% | 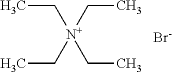 | C₈H₂₀BrN |
| Tetraethylammonium chloride for electrochemical analysis, ≥99.0% | 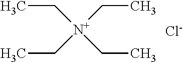 | C₈H₂₀ClN |
| Tetraethylammonium chloride hydrate | 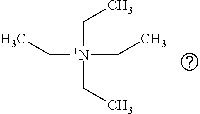 | C₈H₂₀ClN•xH₂O |
| Tetraethylammonium chloride monohydrate ≥ 98.0% | 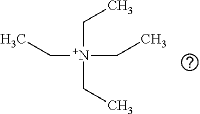 | C₈H₂₀ClN•H₂O |
| Tetraethylammonium cyanate technical | 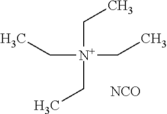 | C₉H₂₀N₂O |
| Tetraethylammonium cyanide purum, ≥95% (AT) | 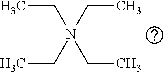 | C₉H₂₀N₂ |
| Tetraethylammonium cyanide 94% | 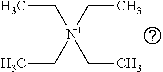 | C₉H₂₀N₂ |
| Tetraethylammonium hexafluorophosphate for electrochemical analysis, ≥99.0% | 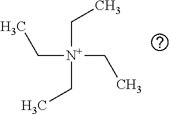 | C₈H₂₀F₆NP |
| Tetraethylammonium hexafluorophosphate 99% | 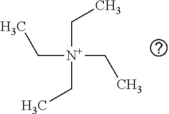 | C₈H₂₀F₆NP |
| Tetraethylammonium hydrogen sulfate ≥ 99.0% (T) | 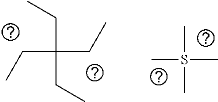 | C₈H₂₁NO₄S |
| Tetraethylammonium hydrogen sulfate ≥ 98.0% (T) | 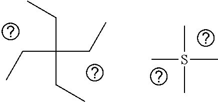 | C₈H₂₁NO₄S |
| Tetraethylammonium iodide puriss., ≥98.5% (CHN) | 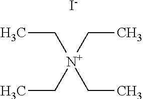 | C₈H₂₀IN |
| Tetraethylammonium iodide 98% | 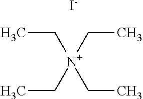 | C₈H₂₀IN |
| Tetraethylammonium nitrate ≥ 98.0% (NT) | 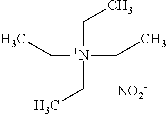 | C₈H₂₀N₂O₃ |
| Tetraethylammonium tetrafluoroborate for electrochemical analysis, ≥99.0% | 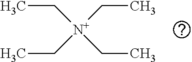 | C₈H₂₀BF₄N |
| Tetraethylammonium tetrafluoroborate purum, ≥98.0% (T) | 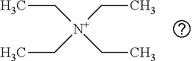 | C₈H₂₀BF₄N |
| Tetraethylammonium tetrafluoroborate 99% | 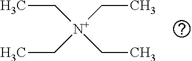 | C₈H₂₀BF₄N |
| Tetraethylammonium p-toluenesulfonate 97% | 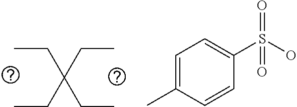 | C₁₅H₂₇NO₃S |
| Tetraethylammonium trifluoromethanesulfonate ≥ 98.0% (T) | 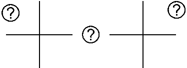 | C₉H₂₀F₃NO₃S |
| Tetraheptylammonium bromide ≥ 99.0% (AT) | 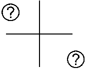 | C₂₈H₆₀BrN |
| Tetraheptylammonium iodide ≥ 99.0% (AT) | 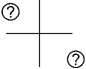 | C₂₈H₆₀IN |
| Tetrahexadecylammonium bromide purum, ≥98.0% (NT) | 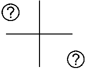 | C₆₄H₁₃₂BrN |
| Tetrahexadecylammonium bromide 98% | 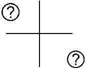 | C₆₄H₁₃₂BrN |
| Tetrahexylammonium benzoate solution ~75% in methanol | 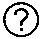 | C₃₁H₅₇NO₂ |
| Tetrahexylammonium bromide 99% | 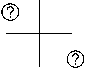 | C₂₄H₅₂BrN |
| Tetrahexylammonium chloride 96% | 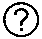 | C₂₄H₅₂ClN |
| Tetrahexylammonium hexafluorophosphate ≥ 97.0% (gravimetric) | 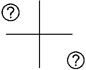 | C₂₄H₅₂F₆NP |
| Tetrahexylammonium hydrogensulfate 98% | 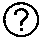 | C₂₄H₅₃NO₄S |
| Tetrahexylammonium hydrogensulfate ≥ 98.0% (T) | 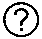 | C₂₄H₅₃NO₄S |
| Tetrahexylammonium iodide ≥ 99.0% (AT) | 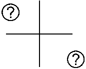 | C₂₄H₅₂IN |
| Tetrahexylammonium tetrafluoroborate ≥ 97.0% | 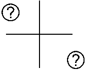 | C₂₄H₅₂BF₄N |
| Tetrakis(decyl)ammonium bromide > 99% (titration) | 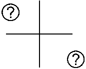 | C₄₀H₈₄BrN |
| Tetrakis(decyl)ammonium bromide ≥ 99.0% (AT) | 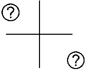 | C₄₀H₈₄BrN |
| Tetramethylammonium acetate technical grade, 90% | 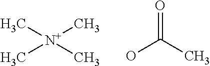 | C₆H₁₅NO₂ |
| Tetramethylammonium benzoate electrochemical grade, ≥98.5% (NT) | 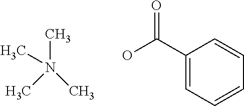 | C₁₁H₁₇NO₂ |
| Tetramethylammonium bis(trifluoromethanesulfonyl)imide 97% | 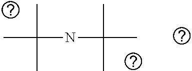 | C₆H₁₂F₆N₂O₄S₂ |
| Tetramethylammoniumbisulfate hydrate ≥ 98.0% (calc. on dry | [symbol] •xH₂O | C₄H₁₃NO₄S•xH₂O |
| substance, T) | | |
| Tetramethylammonium bromide ACS reagent ≥ 98.0% | 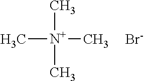 | C₄H₁₂BrN |
| Tetramethylammonium bromide 98% | 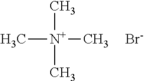 | C₄H₁₂BrN |
| Tetramethylammonium bromide for electrochemical analysis, ≥99.0% | 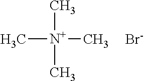 | C₄H₁₂BrN |
| Tetramethylammonium chloride for electrochemical analysis, ≥99.0% | 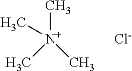 | C₄H₁₂ClN |
| Tetramethylammonium chloride purum, ≥98.0% (AT) | 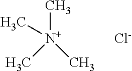 | C₄H₁₂ClN |
| Tetramethylammonium chloride reagent grade, ≥98% | 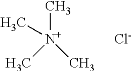 | C₄H₁₂ClN |
| Tetramethylammonium chloride solution for molecular biology | 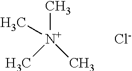 | |
| Tetramethylammonium formate solution 30 wt. % in H₂O, ≥99.99% trace metals basis | 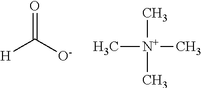 | C₅H₁₃NO₂ |
| Tetramethylammonium hexafluorophosphate ≥ 98.0% (gravimetric) | 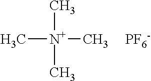 | C₄H₁₂F₆NP |
| Tetramethylammonium hydrogen sulfate monohydrate crystallized, | [symbol] O | C₄H₁₃NO₄S•H₂O |
| ≥98.0% (T) | | |
| Tetramethylammonium hydrogensulfate hydrate 98% | [symbol] O | C₄H₁₃NO₄S•xH₂O |
| Tetramethylammonium iodide 99% | 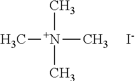 | C₄H₁₂IN |
| Tetramethylammonium nitrate 96% | (CH₃)₄N(NO₃) | C₄H₁₂N₂O₃ |
| Tetramethylammonium silicate solution 15-20 wt. % in H₂O, | [symbol] | C₄H₁₃NO₅Si₂ |
| ≥99.99% trace metals basis | | |
| Tetramethylammonium sulfate hydrate | 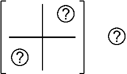 | C₈H₂₄N₂O₄S•xH₂O |
| Tetramethylammonium tetrafluoroborate purum, ≥98.0% (T) | 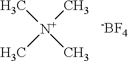 | C₄H₁₂BF₄N |
| Tetramethylammonium tetrafluoroborate 97% | 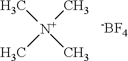 | C₄H₁₂BF₄N |
| Tetramethylammonium tribromide purum, ≥98.0% (AT) | 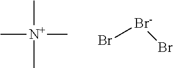 | C₄H₁₂Br₃N |
| Tetraoctadecylammonium bromide purum, ≥98.0% (NT) | 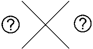 | C₇₂H₁₄₈BrN |
| Tetraoctadecylammonium bromide 98% | 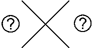 | C₇₂H₁₄₈BrN |
| Tetraoctylammonium bromide purum, ≥98.0% (AT) | 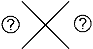 | C₃₂H₆₈BrN |
| Tetraoctylammonium bromide 98% | 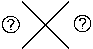 | C₃₂H₆₈BrN |
| Tetraoctylammonium chloride ≥ 97.0% (AT) | 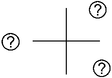 | C₃₂H₆₈ClN |
| Tetrapentylammonium bromide ≥ 99% | 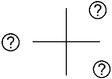 | C₂₀H₄₄NBr |
| Tetrapentylammonium chloride 99% | 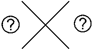 | C₂₀H₄₄ClN |
| Tetrapropylammonium perchlorate ≥ 98.0% (T) | 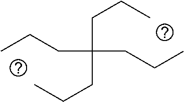 | C₁₂H₂₈ClNO₄ |
| Tetrapropylammonium bromide for electrochemical analysis, ≥99.0% | 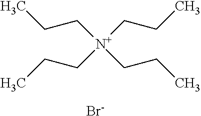 | C₁₂H₂₈BrN |
| Tetrapropylammonium bromide purum, ≥98.0% (AT) | 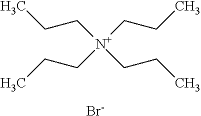 | C₁₂H₂₈BrN |
| Tetrapropylammonium bromide 98% | 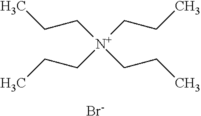 | C₁₂H₂₈BrN |
| Tetrapropylammonium chloride 98% | 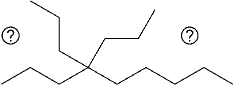 | C₁₂H₂₈ClN |
| Tetrapropylammonium iodide ≥ 98% | 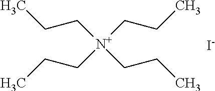 | C₁₂H₂₈IN |
| Tetrapropylammonium tetrafluoroborate ≥ 98.0% | 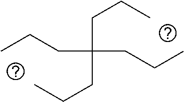 | C₁₂H₂₈BF₄N |
| Tributylammonium pyrophosphate | 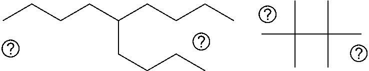 | |
| Tributylmethylammonium bromide ≥ 98.0% | 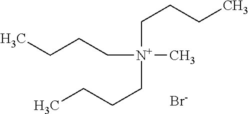 | C₁₃H₃₀BrN |
| Tributylmethylammonium chloride ≥ 98.0% (T) | 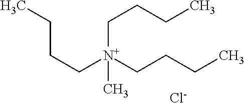 | C₁₃H₃₀ClN |
| Tributylmethylammonium chloride solution 75 wt. % in H₂O | 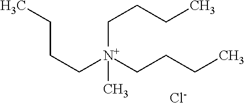 | C₁₃H₃₀ClN |
| Tributylmethylammonium methyl sulfate ≥ 95% | 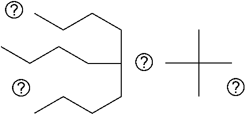 | C₁₄H₃₃NO₄S |
| Tricaprylylmethylammonium chloride mixture of C₈-C₁₀ C₈ is dominant | 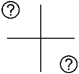 | |
| Tridodecylmethylammonium chloride purum, ≥97.0% (AT) | 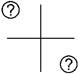 | C₃₇H₇₈ClN |
| Tridodecylmethylammonium chloride 98% | 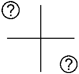 | C₃₇H₇₈ClN |
| Tridodecylmethylammonium iodide 97% | 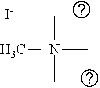 | C₃₇H₇₈IN |
| Triethylhexylammonium bromide 99% | 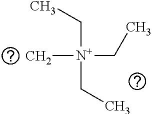 | C₁₂H₂₈BrN |
| Triethylmethylammonium bromide ≥ 99.0% | 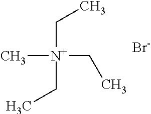 | C₇H₁₈BrN |
| Triethylmethylammonium chloride 97% | 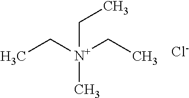 | C₇H₁₈ClN |
| Trihexyltetradecylammonium bromide ≥ 97.0% (T) | 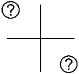 | C₃₂H₆₈BrN |
| Trimethyloctadecylammonium bromide purum, ≥97.0% (AT) | 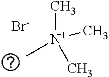 | C₂₁H₄₆BrN |
| Trimethyloctadecylammonium bromide 98% | 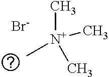 | C₂₁H₄₆BrN |
| Trimethyloctylammonium bromide ≥ 98.0% (AT) | 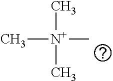 | C₁₁H₂₆BrN |
| Trimethyloctylammonium chloride ≥ 97.0% (AT) | 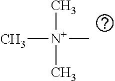 | C₁₁H₂₆ClN |
| Trimethylphenylammonium bromide 98% | 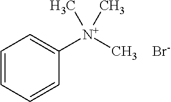 | C₉H₁₄BrN |
| Trimethylphenylammonium chloride ≥ 98% | 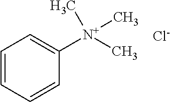 | C₉H₁₄ClN |
| Trimethylphenylammonium tribromide 97% | 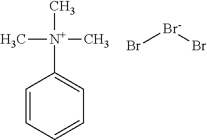 | C₉H₁₄Br₃N |
| Trimethyl-tetradecylammonium chloride ≥ 98.0% (AT) | 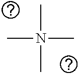 | C₁₇H₃₈ClN |
| (Vinylbenzyl)trimethylammonium chloride 99% | 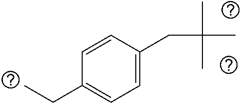 | C₁₂H₁₈ClN |
| N-(Allyloxycarbonyloxy)succinimide 96% | 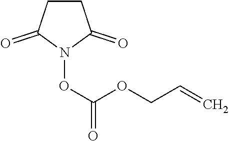 | C₈H₉NO₅ |
| 3-Benzyl-5-(2-hydroxyethyl)-4-methylthiazolium chloride purum, ≥99.0% (AT) | 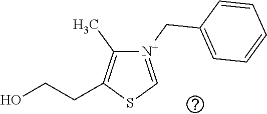 [SMILES: Cc1c(CCO)sc[n+]1Cc1ccccc1] | C₁₃H₁₆ClNOS |
| 3-Benzyl-5-(2-hydroxyethyl)-4-methylthiazolium chloride 98% | 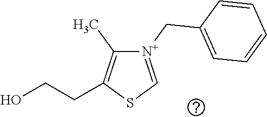 [SMILES: Cc1c(CCO)sc[n+]1Cc1ccccc1] | C₁₃H₁₆ClNOS |
| 1-Butyl-2,3-dimethylimidazolium chloride ≥ 97.0% (HPLC/AT) | 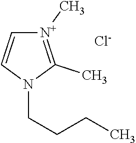 | C₉H₁₇ClN₂ |
| 1-Butyl-2,3-dimethylimidazolium hexafluorophosphate | 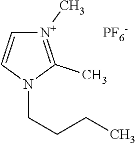 | C₉H₁₇F₆N₂P |
| 1-Butyl-2,3-dimethylimidazolium tetrafluoroborate ≥ 97.0% | 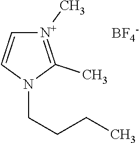 | C₉H₁₇BF₄N₂ |
| 1,3-Didecyl-2-methylimidazolium chloride 96% | 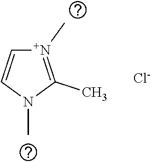 | C₂₄H₄₇ClN₂ |
| 1,1-Dimethyl-4-phenylpiperazinium iodide ≥ 99.0% (AT) | 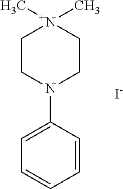 | C₁₂H₁₉IN₂ |
| 1-Ethyl-2,3-dimethylimidazolium ethyl sulfate BASF quality, ≥94.5% (HPLC) | 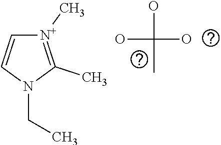 | C₉H₁₈N₂O₄S |
| 3-Ethyl-5-(2-hydroxyethyl)-4-methylthiazolium bromide ≥ 98% | 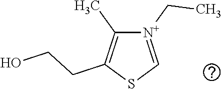 | C₈H₁₄BrNOS |
| Hexadecylpyridinium bromide | 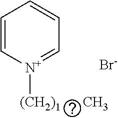 | C₂₁H₃₈BrN |
| Hexadecylpyridinium bromide ≥ 97.0% | 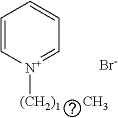 | C₂₁H₃₈BrN |
| Hexadecylpyridinium chloride monohydrate BioXtra, 99.0-102.0% | 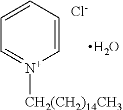 | C₂₁H₃₈ClN•H₂O |
| 5-(2-Hydroxyethyl)-3,4-dimethylthiazolium iodide 98% | 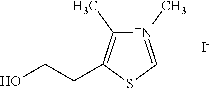 | C₇H₁₂INOS |
| 1-Methylimidazolium hydrogen sulfate 95% | 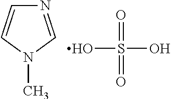 | C₄H₆N₂•H₂SO₄ |
| Methyl viologen dichloride hydrate 98% | 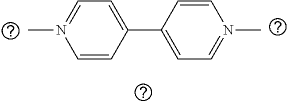 | C₁₂H₁₄Cl₂N₂•xH₂O |
| 1,2,3-Trimethylimidazolium methyl sulfate BASF quality, 95% | 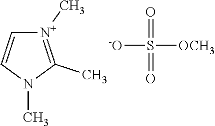 | C₇H₁₄N₂O₄S |
| DL-α-Tocopherol methoxypolyethylene glycol succinate | | |
| DL-α-Tocopherol methoxypolyethylene glycol succinate solution | | |
| 2 wt. % in H2O | | |
| DL-α-Tocopherol methoxypolyethylene glycol succinate solution | | |
| 5 wt. % in H2O | | |
| Aliquat ® HTA-1 High-Temperature Phase Transfer Catalyst, 30- | | |
| 35% in H₂O | | |
| Bis[tetrakis(hydroxymethyl)phosphonium] sulfate solution technical, 70-75% in H₂O (T) | 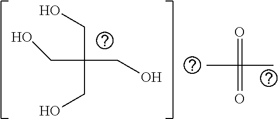 | C₈H₂₄O₁₂P₂S |
| Dimethyldiphenylphosphonium iodide purum, ≥98.0% (AT) | 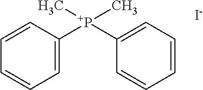 | C₁₄H₁₆IP |
| Dimethyldiphenylphosphonium iodide 98% | 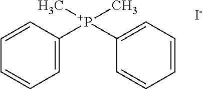 | C₁₄H₁₆IP |
| Methyltriphenoxyphosphonium iodide 96% | 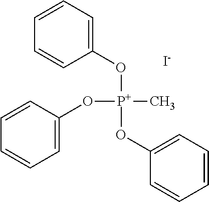 [SMILES: C[P+](Oc1ccccc1)(Oc1ccccc1)Oc1ccccc1 ; [I-]] | C₁₉H₁₈IO₃P |
| Methyltriphenoxyphosphonium iodide technical, ≥96.0% (AT) | 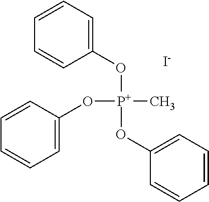 [SMILES: C[P+](Oc1ccccc1)(Oc1ccccc1)Oc1ccccc1 ; [I-]] | C₁₉H₁₈IO₃P |
| Tetrabutylphosphonium bromide 98% | 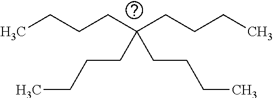 [SMILES: CCCCC(CCCC)(CCCC)CCCC] | C₁₆H₃₆BrP |
| Tetrabutylphosphonium chloride 96% | 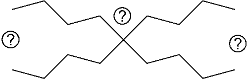 [SMILES: CCCCC(CCCC)(CCCC)CCCC] | C₁₆H₃₆ClP |
| Tetrabutylphosphonium hexafluorophosphate for electrochemical analysis, ≥99.0% | 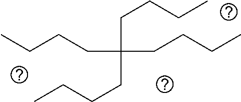 [SMILES: CCCCC(CCCC)(CCCC)CCCC] | C₁₆H₃₆F₆P₂ |
| Tetrabutylphosphonium methanesulfonate ≥ 98.0% (NT) | 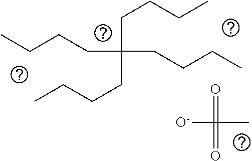 | C₁₇H₃₉O₃PS |
| Tetrabutylphosphonium tetrafluoroborate for electrochemical analysis, ≥99.0% | 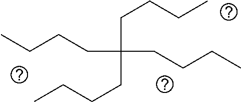 [SMILES: CCCCC(CCCC)(CCCC)CCCC] | C₁₆H₃₆BF₄P |
| Tetrabutylphosphonium p-toluenesulfonate ≥ 95% (NT) | 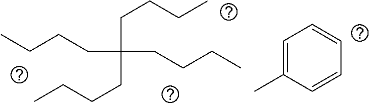 | C₂₃H₄₃O₃PS |
| Tetrakis(hydroxymethyl)phosphonium chloride solution 80% in H₂O | 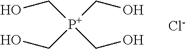 | C₄H₁₂ClO₄P |
| Tetrakis(hydroxymethyl)phosphonium chloride solution technical, ~80% in H₂O | 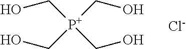 | C₄H₁₂ClO₄P |
| Tetrakis[tris(dimethylamino)phosphoranylidenamino]phosphonium chloride ≥ 98.0% | 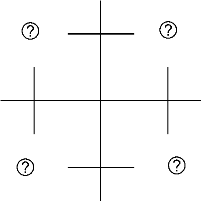 [SMILES: CC(C)(C)CC(CC(C)(C)C)(CC(C)(C)C)CC(C)(C)C] | C₂₄H₇₂ClN₁₆P₅ |
| Tetramethylphosphonium bromide 98% | 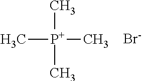 | C₄H₁₂BrP |
| Tetramethylphosphonium chloride 98% | 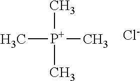 | C₄H₁₄ClP |
| Tetraphenylphosphonium bromide 97% | 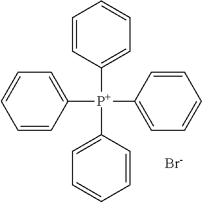 [SMILES: c1ccc([P+](c2ccccc2)(c2ccccc2)c2ccccc2)cc1 ; [Br-]] | C₂₄H₂₀BrP |
| Tetraphenylphosphonium chloride for the spectrophotometric det. of Bi, Co, ≥97.0% | 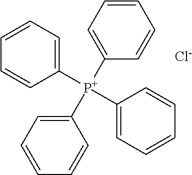 [SMILES: c1ccc([P+](c2ccccc2)(c2ccccc2)c2ccccc2)cc1 ; [Cl-]] | C₂₄H₂₀ClP |
| Tetraphenylphosphonium chloride 98% | 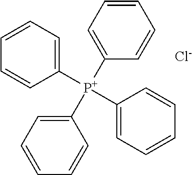 [SMILES: c1ccc([P+](c2ccccc2)(c2ccccc2)c2ccccc2)cc1 ; [Cl-]] | C₂₄H₂₀ClP |
| Tributylhexadecylphosphonium bromide 97% | 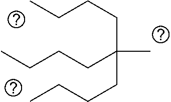 | C₂₈H₆₀BrP |
| Trihexyltetradecylphosphonium bis(2,4,4-trimethylpentyl) phosphinate ≥95.0% | 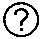 | C₄₈H₁₀₂O₂P₂ |
| Trihexyltetradecylphosphonium bromide ≥ 95% | 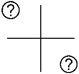 | C₃₂H₆₈BrP |
| Trihexyltetradecylphosphonium chloride ≥ 95.0% (NMR) | 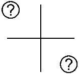 | C₃₂H₆₈ClP |
| Trihexyltetradecylphosphonium dicyanamide ≥ 95% | 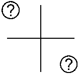 | C₃₄H₆₈N₃P |
| ALKANOL ® 6112 surfactant | | |
| Adogen ® 464 | | |
| Brij ® 52 main component: diethylene glycol hexadecyl ether | | |
| Brij ® 52 average Mₙ~330 | | |
| Brij ® 93 average Mₙ~357 | | |
| Brij ® S2 main component: diethylene glycol octadecyl ether | | |
| Brij ® S 100 average Mₙ~4,670 | | |
| Brij ® 58 average Mₙ~1124 | | |
| Brij ® C10 average Mₙ~683 | | |
| Brij ® L4 average Mₙ~362 | | |
| Brij ® O10 average Mₙ~709 | | |
| BRIJ ® O20 average Mₙ~1,150 | | |
| Brij ® S10 average Mₙ~711 | | |
| Brij ® S20 | | |
| Ethylenediamine tetrakis(ethoxylate-block-propoxylate) | | |
| tetrol average Mₙ~7,200 | | |
| Ethylenediamine tetrakis(ethoxylate-block-propoxylate) | | |
| tetrol average Mₙ~8,000 | | |
| Ethylenediamine tetrakis(propoxylate-block-ethoxylate) | | |
| tetrol average Mₙ~3,600 | | |
| IGEPAL ® CA-520 average Mₙ~427 | | |
| IGEPAL ® CA-720 average Mₙ~735 | | |
| IGEPAL ® CO-520 average Mₙ 441 | | |
| IGEPAL ® CO-630 average Mₙ 617 | | |
| IGEPAL ® CO-720 average Mₙ~749 | | |
| IGEPAL ® CO-890 average Mₙ~1,982 | | |
| IGEPAL ® DM-970 | | |
| MERPOL ® DA surfactant 60 wt. % in water: isobutanol (ca. | | |
| 50:50) | | |
| MERPOL ® HCS surfactant | | |
| MERPOL ® OJ surfactant | | |
| MERPOL ® SE surfactant | | |
| MERPOL ® SH surfactant | | |
| MERPOL ® A surfactant | | |
| Poly(ethylene glycol) sorbitan tetraoleate | | |
| Poly(ethylene glycol) sorbitol hexaoleate | | |
| Poly(ethylene glycol) (12) tridecyl ether mixture of C₁₁ to C₁₄ | | |
| iso-alkyl ethers with C₁₃ iso-alkyl predominating | | |
| Poly(ethylene glycol) (18) tridecyl ether mixture of C₁₁ to C₁₄ | | |
| iso-alkyl ethers with C₁₃ iso-alkyl predominating | | |
| Polyethylene-block-poly(ethylene glycol) average Mₙ~575 | | |
| Polyethylene-block-poly(ethylene glycol) average Mₙ~875 | | |
| Polyethylene-block-poly(ethylene glycol) average Mₙ~920 | | |
| Polyethylene-block-poly(ethylene glycol) average Mₙ~1,400 | | |
| Sorbitan monopalmitate | | |
| 2,4,7,9-Tetramethyl-5-decyne-4,7-diol ethoxylate average Mₙ 670 | | |
| 2,4,7,9-Tetramethyl-5-decyne-4,7-diol, mixture of (±) and | | |
| meso 98% | | |
| Triton ™ N-101, reduced | | |
| Triton ™ X-100 | | |
| Triton ™ X-100 reduced | | |
| Triton ™ X-114, reduced reduced, ≥99% | | |
| Triton ™ X-114, reduced reduced | | |
| Triton ™ X-405, reduced reduced | | |
| TWEEN ® 20 average Mₙ~1,228 | | |
| TWEEN ® 40 viscous liquid | | |
| TWEEN ® 60 nonionic detergent | | |
| TWEEN ® 85 | | |

[symbol]

indicates data missing or illegible when filed

1.-34. (canceled)

35. An apparatus comprising:
a radiation source;
an irradiated solution including a metallic compound, a C₁₂TAB or C₁₆TAB surfactant, and an acid, the irradiated solution irradiated by a low dose of ionizing radiation from the radiation source; and
a container to hold the irradiated solution.

36. The apparatus of claim 35, wherein the irradiated solution has a color and the color has a color intensity that increases with an increase in the low dose of ionizing radiation.

37. The apparatus of claim 35, wherein the irradiated solution is formed from a solution having a substantially linear response to the low dose of ionizing radiation.

38. The apparatus of claim 35, wherein the low dose of ionizing radiation has a value of between about 0.5 Gy and about 2.0 Gy.

39. The apparatus of claim 35, wherein the low dose of ionizing radiation has a value of between about 1.7 Gy and about 2.2 Gy.

40. The apparatus of claim 35, wherein the low dose of ionizing radiation has a value of between about 3.0 Gy and about 10.0 Gy.

41. The apparatus of claim 35, wherein the irradiated solution includes a plasmonic nanoparticle.

42. The apparatus of claim 37, wherein the C₁₂TAB or C₁₆TAB surfactant has a concentration and the solution has a color response and modifying the concentration of the surfactant changes the color response of the solution in response to changes to the low dose of ionizing radiation.
